(19) United States
(12) Patent Application Publication
(10) Pub. No.: US 2026/0191795 A1
(43) Pub. Date: Jul. 9, 2026

(54) IMPROVED EXTRACELLULAR VESICLES FROM NON-MAMMALIAN CELLS, METHODS OF ENHANCED PRODUCTION AND USES THEREOF

(71) Applicants: Nils Heinrich THOENNISSEN, Gruenwald (DE); Bernd DIETEL, Lachen (CH)

(72) Inventors: Nils Heinrich THOENNISSEN, Gruenwald (DE); Bernd DIETEL, Lachen (CH)

(21) Appl. No.: 19/132,648

(22) PCT Filed: Nov. 23, 2023
(86) PCT No.: PCT/EP2023/082877

§ 371 Date: May 23, 2025

(30) Foreign Application Priority Data
Nov. 24, 2022 (EP) 22209490.6

(51) Int. Cl. A61K 9/50; C12N 1/205; C12N 5/04; C12N 13/00; C12N 15/113; C12R 1/225

(52) U.S. Cl. CPC A61K 9/5068; C12N 1/205; C12N 5/04; C12N 13/00; C12N 15/113; C12N 2310/141; C12R 2001/225

(57) ABSTRACT

The present invention generally relates to a method for enhancing the production of extracellular vesicles (EVs) from non-mammalian cells, the method comprising: (a) subjecting the cells to an acoustic and/or electromagnetic stimulation; and (b) optionally, collecting the EVs produced from the cells after the stimulation. The present invention relates further to a method of fermenting non-mammalian cells. Moreover, the present invention relates to EVs obtainable by the methods of the invention, to the use of the EVs of the invention in the manufacture of a beverage, a non-beverage food, a dietary supplement, a drug and/or a cosmetic or personal care product, as well as to the EVs of invention for use as a medicament and in the prevention and/or treatment of a pathological condition selected from the group consisting of a cancerous disease, a cardiovascular disease, a neurodegenerative disease, a metabolic disease, an inflammatory disease, an immune disease and an infectious disease.

The present invention generally relates to a method for enhancing the production of extracellular vesicles (EVs) from non-mammalian cells, the method comprising: (a) subjecting the cells to an acoustic and/or electromagnetic stimulation; and (b) optionally, collecting the EVs produced from the cells after the stimulation. The present invention relates further to a method of fermenting non-mammalian cells. Moreover, the present invention relates to EVs obtainable by the methods of the invention, to the use of the EVs of the invention in the manufacture of a beverage, a non-beverage food, a dietary supplement, a drug and/or a cosmetic or personal care product, as well as to the EVs of invention for use as a medicament and in the prevention and/or treatment of a pathological condition selected from the group consisting of a cancerous disease, a cardiovascular disease, a neurodegenerative disease, a metabolic disease, an inflammatory disease, an immune disease and an infectious disease.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Extracellular vesicles (EVs) are a heterogeneous group of nanoscale lipid bilayer-delimited particles that are naturally secreted by cells into the extracellular space and, unlike a cell, cannot replicate. They can be broadly classified into the three main subtypes “exosomes”, “microvesicles (MVs)” and “apoptotic bodies”, which are differentiated based upon their biogenesis, release pathway, size, content, and function. They carry a cargo of (poly) peptides, nucleic acids (DNA, RNA, including mRNA and many types of small non-coding RNAs, such as microRNAs (miRNAs), Piwi-interacting RNA (piRNA), small nucleolar RNA (snoRNA)), lipids, metabolites, and/or even organelles originating from the parent cells from which they are released. Evidence has been brought forward that EVs are released from nearly all living cells, including those from plants, animals and microbes. Whereas it was initially thought that EVs were simply a mechanism of eliminating waste (including protein aggregates and damaged organelles) from the cell to maintain homeostasis, a growing body of evidence has meanwhile been accumulated confirming that EVs-by shuttling molecular information in the form of their cargoes from donor cells to recipient cells-function as mediators of intercellular communication, cell maintenance, and tumor progression and thereby act as important regulators of normal physiology as well as pathological processes.

Growing interest has therefore been devoted towards an exploitation of EVs, pre-eminently exosomes, in the diagnosis, prognosis and treatment of disease, especially of cancer. For example, as exosomes are found in nearly all body fluids, increasing studies suggest a great promise for exosomes to serve as novel biomarkers in exosome-based liquid biopsy of cancer which through analysis of their cargo (e.g., DNA or RNA) may allow profiling of a patient's tumor activity and monitoring disease progression non-invasively and in real-time (Yu et al., 2022). Moreover, due to their inherent proficiency in delivering molecules to cells and even protect them from enzymatic degradation, strategies have been focused on the use of exosomes as delivery vehicles (i.e., nanocarriers) for both, drugs, such as antitumor agents, and diagnostic agents, such as contrast agents (Bhatti et al., 2019; Zhao et al., 2022; Zheng et al., 2020; Lorenc et al., 2020).

Although in the last two decades, research has predominantly been devoted on the characterization of EVs of animal origin with only a significantly smaller proportion of studies conducted independently on EVs from other sources, such as plants and microbes, it has more recently strikingly been demonstrated that EVs can transmit their cargo across different species, even in a cross-kingdom manner, suggesting EVs to participate in cross-species and cross-kingdom communication (Urzi et al., 2021; Urzi et al., 2022).

These findings, in conjunction with the growing awareness of the health-promoting attributes of natural substances found as constituents of EVs from plants and microbes, have triggered a great interest in the utilization of plant- or microbe-derived EVs as pharmaceutical, nutritional, or cosmetic agents (see, e.g., the review articles on plant-derived EVs by Karamanidou et al., 2021; or Cai et al., 2022).

However, despite the recent advancements in the general understanding of the biological functions of EVs and the widely recognized potential for their exploitation in health and disease, their intrinsically low abundance and the absence of scalable production methods remain a major challenge to their more widespread application. Accordingly, there is an unmet need in the art for methods that provide an enhanced production of extracellular vesicles (EVs) from non-mammalian cells, in particular from plant cells and/or microbes.

Moreover, there is also a need in the art for EVs derived from plant cells and/or microbes having improved therapeutic and/or cosmetic properties relative to naturally occurring EVs, as well as for novel methods of generation of such EVs.

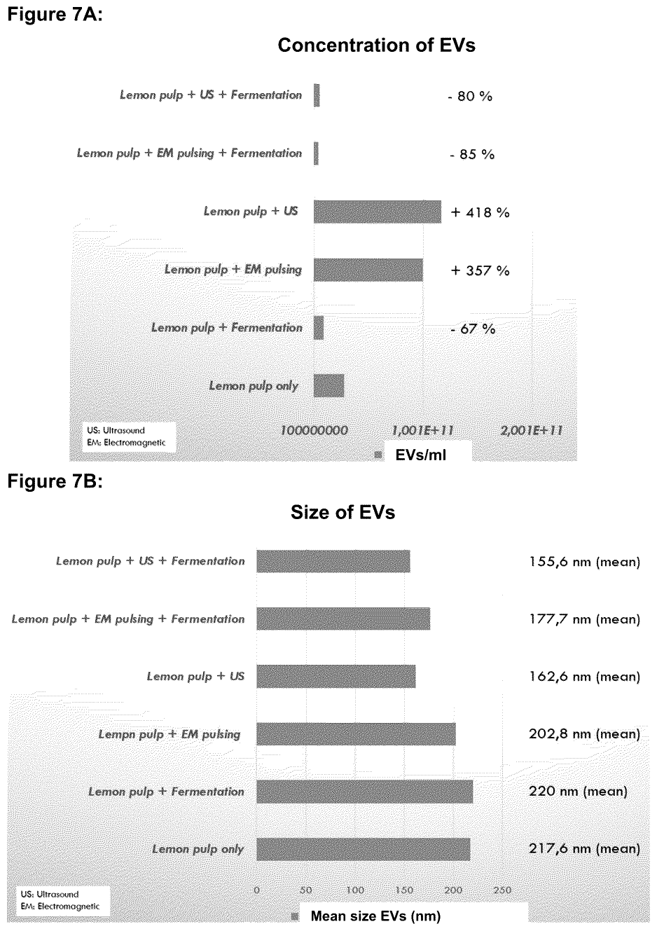

The present invention addresses these and other needs and provides related advantages as well. As demonstrated herein, it was surprisingly found by the present inventors that an exposure of the cells from which the EVs are to be obtained, in the present exemplified case cells from, e.g., lemon fruits or ginger rhizome, to an acoustic or electromagnetic stimulation results in a significant enhancement of the production of EVs (i.e., their intracellular generation and subsequent secretion into the extracellular medium). In particular, as shown herein, it was unexpectedly and advantageously found by the present inventors that upon a respective stimulation of the cells, the number of EVs produced by these cells increased by a factor of at least about 3.6 (357%) or even about 4.2 (418%); see Examples 2 and 3 and FIG. 7A.

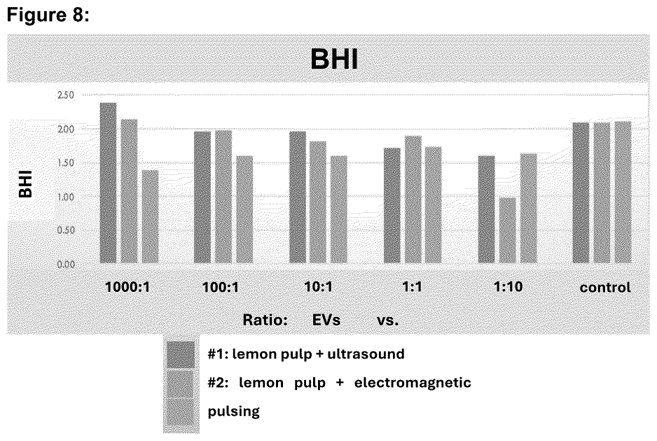

In addition, it was also surprisingly found by the present inventors that the EVs obtained by the herein disclosed inventive methods have a superior capacity to improve the bioenergetic profile of target cells. As demonstrated in Example 7 and corresponding FIG. 8, when applied to human peripheral blood mononuclear cells (PBMCs), the EVs of the invention led to an improvement of the bioenergetic function of the mitochondria as assessed by a determined increase of the Bioenergetic Health Index (BHI).

Moreover, it was furthermore identified by the inventors that the EVs obtained by the herein disclosed inventive methods are characterized by an altered content, in particular an increased content of miRNAs and different relative abundance of individual miRNAs (see Example 9).

Accordingly, the invention relates in a first aspect to a method for (or “of”) enhancing the production of extracellular vesicles (EVs) from non-mammalian cells, the method comprising: (a) subjecting the cells to an acoustic and/or electromagnetic stimulation; and (b) optionally, collecting the EVs produced from the cells after the stimulation.

The term “extracellular vesicles” (EVs), as used herein, refers broadly to all sorts of secreted vesicles composed of an outer lipid-bilayer membrane enclosing an aqueous core comprising cargo such as, without limitation, (poly) peptides, nucleic acids (e.g., DNA, RNA, mRNA and/or microRNA(s)), lipids, and/or metabolites. It is hence understood that the term “EVs” in accordance with its broadest meaning also includes exosomes, microvesicles (MVs) and apoptotic bodies, and may as well encompass vesicle- or exosome-like nanoparticles and nano-vesicles and the like. In preferred embodiments, the term “extracellular vesicles” refers to “exosomes”.

The term “(poly) peptide”, as used herein, refers to a linear polymer of amino acid residues linked by peptide bonds in a specific sequence and embraces both, the group of “polypeptides” and the group of “peptides”. The group of “polypeptides”, as interchangeably used herein with the term “protein”, consists of molecules with more than 30 amino acids, which is in distinction to the group of “peptides” which consists of molecules with up to 30 amino acids. The group of “peptides” also refers to fragments of proteins of a length of 30 amino acids or less. (Poly) peptides may further form dimers, trimers and higher oligomers, i.e., consisting of more than one (poly) peptide molecule. (Poly) peptide molecules forming such dimers, trimers etc. may be identical or non-identical. The corresponding higher order structures are, consequently, termed homo- or heterodimers, homo- or heterotrimers etc. Homo- or heterodimers etc. also fall under the definition of the term “(poly) peptide”. The term “(poly) peptide” also refers to chemically or post-translationally modified peptides and polypeptides.

The term “nucleic acid”, as referred to herein, can be interchangeably used with the terms “nucleic acid molecule” or “polynucleotide”, in accordance with the present invention, includes DNA, such as cDNA or genomic DNA, and RNA. It is understood that the term “RNA”, as used herein, comprises all forms of RNA, including mRNA and many types of small non-coding RNAs, such as microRNAs (miRNAs), Piwi-interacting RNA (piRNA), small nucleolar RNA (snoRNA)). Moreover, it is also to be understood that within any of the nucleotide sequences disclosed or otherwise referred to herein, U may alternatively be T or T may alternatively be U. For example, the skilled person is aware that DNA has thymine as the complementary nucleotide to adenine, whereas in RNA, uracil (U) replaces thymine (T) as the complementary nucleotide to adenine. Thus, as used herein, an RNA sequence may alternatively be expressed by a nucleotide sequence which, instead of a U, comprises a T. For example, for sequencing of an RNA, the RNA is typically extracted from the sample and then converted into complementary DNA (cDNA) using reverse transcription. The obtained cDNA which is subsequently subjected to sequencing hence comprises Ts instead of Us as complementary nucleotide to A. Accordingly, when assessing a sample for the presence of a certain RNA (such as an miRNA) by sequencing of the RNA, the sequence read corresponding to said RNA may comprise Ts instead of Us.

“Exosomes” are formed by an endosomal route, specifically by inward budding of the limiting membrane of early endosomes, which mature into multivesicular bodies (MVBs) during the process. Early endosomes, which originate from inward budding of the cell's plasma membrane, and MVBs are involved in the endocytic and trafficking functions of the cell's material, including protein sorting, recycling, storage, transport, and release. MVBs are eventually either sent to the lysosome to be degraded along with all of its components or fused with the cell's plasma membrane to release its content, including exosomes, into the extracellular space.

“Microvesicles” (MVs), also commonly referred to as “ectosomes” or “microparticles” (MP), are released from the surface of cells. They are formed by direct outward budding, or pinching, of the cell's plasma membrane. The size of MVs typically range from 100 nm up to 1000 nm in diameter. The route of MV formation is not well understood, however, it is thought to require cytoskeleton components, such as actin and microtubules, along with molecular motors (kinesins and myosins), and fusion machinery (SNAREs and tethering factors).

“Apoptotic bodies” are released through blebbing by cells undergoing programmed cell death into the extracellular space. They are reported to range in size from 50 nm up to 5000 nm in diameter, with the size of most apoptotic bodies tending to be on the larger side. These bodies form by a separation of the cell's plasma membrane from the cytoskeleton as a result of an increased hydrostatic pressure after the cell contracts.

The term “non-mammalian cell”, as used herein, means in its broadest sense a cell which is derived from an organism other than a mammal, including, without limitation, prokaryotes (archaea, bacteria) as well as unicellular and multicellular eukaryotes, such as protists, fungi, plants and non-mammalian animals (i.e., an animal other than a mammal).

In preferred embodiments, however, the non-mammalian cells are non-animal cells, more preferably plant cells and/or eukaryotic or prokaryotic (preferably, prokaryotic) microorganisms. In even more preferred embodiments, the non-mammalian cells consist essentially of, or consist of plant cells.

The term “consist essentially of”, as used herein, means contents of at least 95%, preferably, at least 99%, most preferably 99.9% of the total number (or volume or weight) of the cells being present. The term, however, is not intended to exclude the accidental or deliberate inclusion of traces of other non-mammalian cells which may be present. For example, it is well known that many plant materials, a prominent example of which being grapes, naturally comprise on their surfaces trace amounts of microbes, such as certain yeasts, which presence will not be detrimental to the herein envisaged purposes and technical effects.

The skilled person will understand, in view of the herein disclosed purposes and technical effects, that the non-mammalian cells, for being suitably employed in the methods of the present invention, are living (i.e., viable/alive) cells having intact, or at least substantially intact, plasma membranes, and which thus are capable of producing EVs. The term “non-mammalian cells” is thus intended to also refer in its broadest meaning to protoplasts and spheroplasts (i.e., two altered forms of plant or microbial cells from which the cell wall has been completely or partially removed). In other preferred embodiments, however, the term “non-mammalian cells” does not include protoplasts and/or spheroplasts.

Means and methods for differentiating viable and non-viable non-mammalian cells, such as plant and/or microbial cells, are known in the art and may be readily employed by the skilled artisan for selecting cells which are suitable for the herein disclosed purposes. For example, for assessing the (non-) viability of plant cells, the Plant Cell Viability Assay Kit from Sigma-Aldrich may be employed which utilizes a dual color fluorescent staining system to highlight viable and non-viable cells. These cells can be distinguished by the presence of intracellular esterase activity which is assayed through the enzymatic hydrolysis of fluorescein diacetate or related compounds, such as carboxyfluorescein or calcein acetoxymethyl (calcein AM). These lipophilic compounds are membrane-permeable and non-fluorescent. In the plant cell, they are hydrolyzed to highly polar fluorescent compounds. Because of their polar nature, these compounds are unable to diffuse across the plasma membrane and are retained within viable cells, producing an intense green fluorescence within the cytoplasm. This procedure has been used to stain intact plant tissue, callus tissue, cell suspension culture, and protoplasts. For assaying the (non-) viability of microorganisms, such as bacteria or yeasts, a suitable (liquid or solid or semi-solid) culture medium may be inoculated with the microorganism to be tested followed by an incubation at culture conditions known to be suitable for culturing of that microorganism and the viability of the microorganism may then be inferred from an observable growth (e.g., in the formation of a colony) on a solid or semi-solid medium (e.g., an agar plate) or increase in the optical density of a liquid culture medium.

As will be readily understood by the skilled person, the non-mammalian cells are preferably a population of (preferably isolated) non-mammalian cells that are suspended in a maintenance or culture medium, typically an aqueous solution. The skilled person will appreciate that, dependent on the specific kind of cells to be employed, e.g., in cases where the non-mammalian cells are from fruits (e.g., citrus, melon, grapes) or other natural raw material having naturally a high water content, the medium may preferably be derived from the same origin as the non-mammalian cells. For instance, in the herein disclosed examples, the non-mammalian cells employed were from lemon fruits and the method was conducted directly on the homogenized lemon pulp, i.e., wherein intact lemon cells were comprised in the natural fruit juice originating from a fraction of cells that were disrupted by the homogenization. The skilled person will understand that employing a maintenance or culture medium which is derived from the same or a similar source as the (or some of) the non-mammalian cells will provide an extracellular milieu which closely resembles that of the cells in their natural environment, and thus provides most suitable conditions (i.e., in terms of pH, osmotic pressure and nutritional constituents) for the cells to survive and a medium into which the generated EVs can be released; and that, however, in other instances, e.g., wherein the non-mammalian cells are from natural raw material typically having a rather low water content, it may be productive to provide a medium, such as an aqueous solution or simply water, wherein the cells are suspended.

In the method according to the first aspect of the invention, the non-mammalian cells are subjected to an acoustic and/or electromagnetic stimulation.

The terms “acoustic stimulation”, “acoustic wave stimulation”, “acoustic energy stimulation”, “acoustic wave energy stimulation” or “acoustic irradiation”, as interchangeably used herein, refer in their broadest sense to any form of an acoustic (i.e., sound) energy or wave which may be provided by a devise, i.e., an acoustic wave generator, and transmitted to the cells for the sake of effecting their stimulation for effecting an enhancement of the production of EVs. Acoustic waves (i.e., sound waves, preferably ultrasound waves) are predominantly longitudinal waves (same direction of vibration as the direction of propagation) that result from an oscillation of pressure that travels through a solid, liquid or gas in a wave pattern. These waves show numerous characteristics including wavelength, frequency, period and amplitude. Suitable devices for the generation and provision of “acoustic wave energy” which may readily be employed for the herein disclosed purposes are available from various commercial suppliers. For example, the Agilent Generator 33220A (Agilent Technologies, Inc., Santa Clara, CA, US) as was used in the herein disclosed examples. Another commercially available device is, e.g., the Bioruptor® Plus from Diagenode Inc. USA.

In preferred embodiments, the acoustic stimulation is a stimulation by ultrasound.

The terms “ultrasound”, “ultrasonication” or “sonication”, as interchangeably used herein refer, in accordance with their common meaning to acoustic waves (a form of pressure waves) with frequencies above the audible limit of human hearing (≥20 KHz up to several GHZ) which need a medium to travel through (unlike light or electromagnetic waves). Unlike EM waves which are transverse waves, acoustic waves propagate mostly longitudinally in gases or liquid. In solids, transversal waves due to shear stress have been found to additionally occur as well. In general, ultrasound waves possess physical properties, such as attenuation, reflection, refraction, amplification, absorption, and scattering, that are inherent in any wave. In practice, ultrasound is typically provided by an ultrasonic generator containing a piezoelectric transducer, which is capable of converting an electrical signal into mechanical pressure waves. These pressure waves cause, when passing, local oscillatory motion of particles through the transmitting medium which results in a local density change in the medium (succession of compression and decompression events). The applied acoustic pressure (measured in Pa) is directly related to the amount of energy received by the targeted tissue. Biological effects induced by ultrasound application can be influenced by varying different parameters such as “mode” (continuous or pulsed), “frequency”, “intensity” and “exposure time”. For example, ultrasound can be applied in a continuous mode or discontinuous mode (so-called pulsed mode). A continuous application of ultrasound for a certain period of time of exposure may lead to a sample heating. In cases where heating is undesired, continuous waves can be broken down into several ultrasound pulses, so that energy dissipation occurs between pulses. Pulses can vary in length and are repeated periodically at a given frequency (repetition per second), which determines the duty-cycle of the ultrasound application.

The most common parameter to describe ultrasound waves is the frequency which is typically expressed as the ratio of speed and wavelength. Ultrasound may generally be applied at low (20-200 kHz), medium (0.7-3 MHz), or high frequency (>3 MHz).

The acoustic intensity, represented as the spatial-peak temporal-average intensity (ISPTA; units of W/cm²), represents the amount of energy delivered to the desired location and is defined as the ratio between the amount of power carried by the acoustic wave and the surface on which it is applied. ISPTA indicates the averaged fraction of the acoustic intensity per second and is derived by spatial-peak pulse-average intensity (ISPPA) multiplied by duty cycle (indicating the fraction of the sonication duration per second). ISPPA is calculated by measuring the pressure of the sound waves (in pascals) using a hydrophone. When operating in pulsed mode, the duty cycle is determined by pulse duration multiplied by pulse repetition frequency. When operating in continuous wave (CW) mode, the duty cycle is 1 (or 100%). In other words, the term “duty cycle” refers to the percentage of time that a pulsed ultrasound wave is on (e.g., a 50% duty cycle means that a pulsed wave is on 50% of the time). At a duty cycle of 100% (also called a continuous duty cycle), the pulsed wave is on 100% of the time. The intensity of ultrasound is commonly classified into two categories: low intensity ultrasound (ranging from 0.125-3 W/cm²); and high intensity ultrasound (ranging from 3 to several thousand W/cm²). Moreover, in general, the intensity is the power of the ultrasound energy and drives temperature effects: the higher intensity and thus the power, the shorter the treatment time to produce a thermal effect. The skilled person will be aware of this latter correlation and thus, when applying ultrasound in the method of the invention, be able to select suitable parameters/settings to avoid an unintentional induction of hyperthermia and consequently irreversible damage of the cells.

Ultrasound has become a widely used technique in the biological and medical fields, as well as in the food industry.

For example, in the biological or biomedical area, ultrasound is a common method of choice for effecting a disruption of cellular membranes for the purpose of extraction of intracellular contents, such as recombinant proteins expressed in microbial expression hosts. Ultrasonication produces cavitation when acoustic power inputs are sufficiently high, allowing for microbubbles at nucleation sites. The bubbles grow during the rarefying phase of the sound wave and then collapse during the compression phase. On collapse, a violent shock wave passes through the medium. The entire process of gas bubble nucleation, growth and collapse due to the action of intense sound waves is called cavitation. When it comes to extraction, the use of ultrasound or sonication for extraction via breaking cell membranes has the advantage of reducing considerably the extraction time and increasing the extract yield. The application of ultrasound at such higher frequencies can disrupt the cell wall structure and accelerate diffusion through membranes; thus, the cell lyses and hence facilitates the release of cell contents. However, in the present case, the skilled person, in light of the herein disclosed technical effects and purposes, will understand that, in connection with the method according to the invention, if the stimulation of the non-mammalian cells were to be conducted by application of ultrasound, the ultrasound parameters are to be selected such that cells lysis is either (preferably) entirely or at least essentially avoided. The term “essentially avoided” with respect to cell lysis is intended to mean that, with increasing preference, at least 80%, at least 85%, at least 90%, at least 95%, at least 98%, at least 99%, or at least 99.9% and most preferably at least 100% of the cells (or of the amount, i.e., mass or number of cells) which are (is) present prior to an ultrasound stimulation are still present in intact (undisrupted) form after the ultrasound stimulation has been concluded.

In the medical area, ultrasound, besides its routine application in diagnostic imaging (i.e., known as sonography or diagnostic medical sonography), is being used as tool in various drug delivery and other therapeutic applications. For example, ultrasound has been shown to facilitate the delivery of drugs across the skin, promote gene therapy to targeted tissues, deliver chemotherapeutic drugs into tumors and deliver thrombolytic drugs into blood clots. In addition, ultrasound has also been shown to facilitate the healing of wounds and bone fractures (see, e.g., review by Mitragotri et al., 2005).

In the food industry, in particular high-intensity ultrasound, is being used in vast variety of processes to alter, either physically or chemically, the properties of foods, for example to generate emulsions, disrupt cells, promote chemical reactions, inhibit enzymes, tenderize meat and modify crystallization processes (see, e.g., review articles by McClements et al., 1995; Bhargava et al., 2021).

There is also a limited number of very recently reported studies which explored an application of ultrasound in the context of EVs, in particular of mammalian exosomes. For example, Yuana et al. reported that ultrasound in combination with gaseous microbubbles (USMB) can trigger exosome release from cancer cells (Yuana et al., 2017). A subsequent study by Yuana et al. reported the use of USMB for loading drug cargo into human umbilical vein endothelial cells (HUVECs) as a strategy for the generation of EVs as drug-loaded nanocarriers (Yuana et al., 2020). In parallel, Ambattu et al. reported that an increase in exosome production was obtained from two cancer cell lines when exposed to a high-frequency acoustic stimulation (Ambattu et al., 2020). Following these observations, a study by Deng et al. explored the concept of whether an in vivo treatment with ultrasound may provide a means for augmenting exosome release from astrocytes, and thereby the exosome-mediated clearance of amyloid-β (AB) peptide plaques from these brain cells, as a potential novel therapeutic strategy for alleviating AB-neurotoxicity in neurodegenerative disorders, such as Alzheimer's disease. Deng et al. thereby reported, as a proof-of-concept, that an ultrasound stimulation, even without microbubbles, of an in vitro culture of human astrocytes resulted in an enhanced exosome release (Deng et al., 2021).

The terms “electromagnetic stimulation” or “electromagnetic radiation” (EMR), as interchangeably used herein, refer in their broadest sense to a stimulation by any form of electromagnetic energy including, but not limited to, electromagnetic fields (EMFs) and the more specific form of pulsed electromagnetic fields (PEMFs) stimulation, emitted by a devise, i.e., an electromagnetic energy generator or electromagnetic radiation device, capable of providing electromagnetic radiation therefrom, to the cells for the sake of effecting their stimulation that results in an enhancement of the production of EVs.

Electromagnetic radiation (EMR) generally consists of waves of the electromagnetic (EM) field, which propagate through space and carry electromagnetic radiant energy. It includes radio waves, microwaves, infrared, (visible) light, ultraviolet, X-rays, and gamma rays. All of these waves form part of the electromagnetic spectrum. Classically, electromagnetic radiation consists of electromagnetic waves, which are synchronized oscillations of electric and magnetic fields. Electromagnetic radiation or electromagnetic waves are created due to periodic change of electric or magnetic field. Depending on how this periodic change occurs and the power generated, different wavelengths of electromagnetic spectrum are produced. In a vacuum, electromagnetic waves travel at the speed of light, commonly denoted c. In homogeneous, isotropic media, the oscillations of the two fields are perpendicular to each other and perpendicular to the direction of energy and wave propagation, forming a transverse wave. The position of an electromagnetic wave within the electromagnetic spectrum can be characterized by either its frequency of oscillation or its wavelength. Electromagnetic waves of different frequency are called by different names since they have different sources and effects on matter. In order of increasing frequency and decreasing wavelength these are: radio waves, microwaves, infrared radiation, visible light, ultraviolet radiation, X-rays and gamma rays.

The term “pulsed electromagnetic fields (PEMFs) stimulation” or “pulsed EMFs” stimulation, as interchangeably used herein, refers to a specific subtype of EMFs stimulation wherein accumulated electric energy is released in very short intervals (see, e.g., review by Mansourian et al. 2021). PEMF is a widely used modality for the treatment of musculoskeletal disorders, e.g., in orthopedic clinical practices to promote bone healing processes due to its capacity to stimulate extracellular matrix synthesis for bone and cartilage repair (Caliogna et al. 2021), and its therapeutically beneficial effects are currently emerging also for the treatment of other pathological conditions, such as inflammation (Kubat et al., 2015).

In the last decade, several studies have demonstrated the capacity of electromagnetic fields (EMFs) to modulate many fundamental biological functions such as gene expression, cell fate and cell differentiation (Amaroli et al., 2013; Kim et al., 2013). The biological effects of EMFs exposure are thought to be through, inter alia, changes in the gating kinetics of voltage-dependent ion channels (especially for H⁺, K⁺ and Ca²⁺). Moreover, the finding that EMFs exposure can enhance the proliferation and differentiation of stem cells gained particular interest in its utilization in tissue engineering or as treatment modality in regenerative medicine (see, e.g., review article by Hamid et al., 2022). Furthermore, a recent study by Wong et al. explored the effects of a pulsed-electromagnetic fields (PEMFs) exposure on (immortalized) mouse myoblasts (i.e., the embryonic precursors of myocytes (also called muscle cells)). Their reported observation that a brief exposure to PEMF of certain defined directionality resulted in an enhanced release of EVs from these myoblasts led the authors speculate that PEMFs, by causing an activation of the muscle cell secretome response, may be utilized as a means to promote myogenesis, e.g., in regenerative medicine or cultivated meat production (Wong et al. 2022).

However, to the best of the present inventors' knowledge, there has been no prior art suggesting, let alone demonstrating, that a stimulation by an acoustic wave energy and/or electromagnetic radiation provides an enhancement of the production of EVs also from other cells than the above-referred particular forms of mammalian cells (mostly immortalized cell lines or cancer cells), or even from (structurally, genetically, physiologically, functionally and anatomically distinct) non-mammalian cells, such as cells from plants or microbial cells.

In accordance with the method of the first aspect of the invention, the method optionally comprises as step (b) collecting the EVs produced from the cells after the stimulation (i.e., after the acoustic and/or electromagnetic stimulation in step (a)).

The term “collecting”, as used herein in the context of the step of collecting EVs, refers to the physical collection, enrichment, isolation and/or separation of the EVs from the cells, cellular debris and/or other components and/or impurities which may be present, e.g., in the maintenance or culture medium, or reaction vessel (e.g., bioreactor) wherein the cells may be comprised.

A variety of methods for collecting/isolating EVs have been developed and are nowadays routinely employed in the art and each of which alone or in combination may be suitably employed for the present purposes. Exemplary known methods, include, without intention to be limiting, (ultra-) centrifugation-based techniques (such as sequential or differential (ultra-) centrifugation, density gradient centrifugation, rate-zonal centrifugation), size-based techniques (such as ultrafiltration, size-based isolation kits (e.g., the ExoMir™ Kit from Bioo Scientific, Austin, TX, USA; or ExoTIC (exosome total isolation chip) as described by Liu et al. 2017), sequential filtration, size exclusion chromatography, flow field-flow fractionation (FFFF), hydrostatic filtration dialysis (HFD)), immunoaffinity capture-based techniques (such as enzyme-linked immunosorbent assay (ELISA) or magneto-immunoprecipitation), and precipitation-based techniques (using water excluding polymers, such as polyethylene glycol (PEG) or lectin-induced agglutination in cases where the EVs display carbohydrates (e.g., as part of glycol-proteins/-lipids) on their surface), as well as microfluidic-based isolation techniques (reviewed, e.g., by Momen-Heravi et al. 2013; Doyle & Wang 2019; Sunkara et al. 2016).

“Sequential centrifugation”, “consecutive centrifugation” or “repeated centrifugation”, as interchangeably referred to herein, is a frequently used procedure to separate EVs based on their sedimentation rate. In a typical protocol, a maintenance or culture medium or other aqueous suspension comprising the EV-producing cells is placed into a centrifugation tube and subjected to repeated centrifugations, where cells and cellular debris sediment sufficiently quickly at a given centrifugal force for a given time to form a compact “pellet” at the bottom of the centrifugation tube, whereas the EVs remain in solution in the supernatant. After each centrifugation, the supernatant (non-pelleted solution) is removed from the tube and re-centrifuged. “Differential centrifugation”, also commonly known as “differential velocity centrifugation”, is a specific form of sequential centrifugation, wherein each subsequent centrifugation step is performed at an increased centrifugal force and/or time. The finally obtained supernatant contains then the enriched EVs, while being substantially devoid of cells and cellular debris. An even higher degree of purity can, however, if desired, be obtained by combining the sequential, preferably differential centrifugation with one or more further EV-isolation techniques, such as a precipitation-based technique or any one or more other techniques known in the art, examples of which being referred to herein above.

Precipitation of EVs is typically done by introducing a water excluding polymer, such as polyethylene glycol (PEG), into a sample (i.e., a maintenance or culture medium). The PEG polymer then “ties up” the water molecules, causing other particles, such as EVs to precipitate out of the solution. The precipitated EVs can then be pelleted by centrifugation. Besides being quick and simple, and not requiring any expensive equipment, a precipitation-based method can also advantageously be employed for reducing the sample volume in which the EVs are present, i.e., to obtain-dependent on the volume at which the precipitated EVs are finally resuspended-a smaller volume of sample wherein the comprised EVs are enriched at higher concentration as compared to the original sample volume. A possible drawback with this methodology is that the water excluding polymer (e.g., PEG) may also cause precipitation of other non-EV sample components, such as extracellular proteins or protein aggregates, if present. In such instances, it may be helpful to include some sample pretreatment, such as filtration and/or (ultra-) centrifugation, in order to deplete the sample from these impurities and thus to prevent (or at least to substantially reduce) contamination of the final EV preparation. Several commercially available solutions and kits have been developed for isolation of EVs based on precipitation from the maintenance or culture medium, e.g., ExoQuick by System Biosciences (Palo Alto, CA, USA) and Total Exosome Isolation Kit by Thermo Fisher Scientific. The skilled person understands that although these solutions and kits have mostly been developed for the sake of isolation/enrichment of exosomes, they may also suitably be employed for the isolation/enrichment of EVs in general.

In the herein disclosed examples, the collecting in step (b) was conducted by a differential centrifugation comprising three consecutive centrifugation steps, followed by a precipitation of the EVs from the final centrifugation supernatant by using the ExoQuick-TC™ exosome precipitation solution (System Biosciences, USA), and concluded by a re-suspension of the precipitated EVs in phosphate buffered saline (PBS) pH 7.4 for being assessed via Nanoparticle Tracking Analysis (NTA).

Thus, in preferred embodiments, the collecting in step (b) is performed by:
(b-1) centrifugation, preferably by differential centrifugation comprising at least three consecutive centrifugation steps, followed by:
(b-2) precipitation of the EVs from the obtained centrifugation supernatant using a water-excluding polymer, and
(b-3) resuspension of the precipitated EVs in an aqueous buffer, preferably in phosphate buffered saline (PBS) pH 7.4.

In exemplary preferred embodiments, a centrifugation is conducted:
(i) at a centrifugal force in the range of between, with increasing preference, 100×g and 100,000×g, 200×g and 50,000×g, 250×g and 25,000×g, or most preferably 300×g and 12,000×g; and/or
(ii) for a time period of in the range of between, with increasing preference, 5 minutes to 4 hours, 10 minutes to 2 hours, 20 minutes to 1.5 hours, or between 30 min and 1 hour; and/or
(iii) at a temperature in the range of between, with increasing preference, 0.1° C. and 42° C., 1° C. and 40° C., 1° C. and 37° C., 1° C. and 35° C., 1° C. and 30° C., 1° and 25°, 1° C. and 20° C., 1° C. and 15° C., 1° C. and 14° C., 1° C. and 13° C., 1° C. and 12° C., 1° C. and 11° C., 1° C. and 10° C., 1° C. and 9° C., 1° C. and 8° C., 1° C. and 7° C., 2° C. and 6° C., 3° C. and 5° C., most preferably at 4° C.

Further particularly preferred embodiments relating to the collection of the EVs in step (b) are described below.

The term “enhance(s)”, “enhancing”, “enhancement” or other related grammatical forms thereof, refers broadly and in accordance with its meaning commonly ascribed to it in the art to improving the quality and/or amount (i.e., quantity) of something (see, e.g., The Cambridge Dictionary (2023). Cambridge: Cambridge University Press: https://dictionary.cambridge.org/dictionary/english/enhance).

Thus, the term “enhancing the production of extracellular vesicles (EVs)”, as used herein, may refer to promoting, typically improving, augmenting, increasing the quantity of EVs generated and released by the non-mammalian cells, as measurable (e.g., via nanoparticle tracking analysis (NTA)) relative to a control which comprises the same kind and/or number of cells and, if applicable, the identical further constituents, and which has been analogously treated, yet without being subjected to the acoustic and/or electromagnetic stimulation, and optionally, additionally a fermentation.

It is understood that the term “enhancing the production of extracellular vesicles (EVs)”, as used herein, may, besides meaning an improvement (i.e., increase) in terms of the quantity, alternatively, preferably additionally, refers to an improvement of the quality of the obtained EVs. An improvement of the quality may be provided, for example, by an alteration of the internal composition (i.e., content) of the EVs. Thus, in some embodiments, an improvement of the quality of EVs may be provided if any component/constituent(s) thereof which is (are) known or presumed to provide, or to at least contribute to, any positive (e.g., health-promoting) effect is (are) enriched in concentration relative to an EV isolated from a reference sample that has not been subjected to a treatment according to the method according to the first and/or second aspect of the invention (i.e., and/or a corresponding acoustic and/or electromagnetic stimulation, and optionally additionally fermentation). It is thus preferably understood that a reference sample comprises the same kind and/or quantity of cells and, if applicable, the identical further constituents, and has been analogously treated, yet without being subjected to a respective acoustic and/or electromagnetic stimulation, and optionally, additionally a fermentation.

It follows that the term “enhancing the production of extracellular vesicles (EVs)”, as used herein, may alternatively be expressed as “enhancing the production of extracellular vesicles (EVs) by quality and/or quantity” or more concisely “enhancing the quality and/or quantity of extracellular vesicles (EVs)”.

With respect to an enhancement in terms of the quantity, as shown herein, it was surprisingly and advantageously found herein that upon an acoustic or electromagnetic stimulation of the non-mammalian cells, i.e., in the present exemplified case, cells from lemon pulp, the amount (i.e., the quantity/number) of extracellular vesicles produced by these cells increased by a factor of at least about 3.6 (357%) or even about 4.2 (418%), respectively, as determined via nanoparticle tracking analysis (NTA).

Accordingly, in preferred embodiments of the method of the first aspect of the invention, the number of EVs produced is increased by, with increasing preference, at least 10%, at least 20%, at least 30%, at least 40%, at least 50%, at least 60%, at least 70%, at least 80%, at least 90%, at least 100%, at least 120%, at least 140%, at least 160%, at least 180%, at least 200%, at least 220%, at least 240%, at least 260%, at least 280%, at least 300%, at least 320%, at least 340%, at least 360%, at least 380%, or at least 400% as compared to the number of EVs produced by a corresponding control population of the same kind of cells which have not been subjected to the acoustic and/or electromagnetic stimulation, and optionally additionally a fermentation, wherein preferably the number of EVs is assessed by nanoparticle tracking analysis (NTA).

“Nanoparticle tracking analysis (NTA)” is a light-scattering technique that allows for the determination of both, particle size and concentration. The size of the particles is estimated using the Stokes-Einstein equation, where the diffusion coefficient is based on the Brownian motion of particles within the chamber. The laser light is scattered as it interacts with the particles (under Brownian motion) within the chamber, and the scattered light is collected by a microscope that has a camera mounted to it. The camera on top of the microscope captures the movement of particles in a video, and then the NTA software uses the movement of the particles in the video to estimate the particle size and concentration (see, e.g., Doyle & Wang 2019). NTA is prominently used in the art for the characterization of EVs and was also employed in the herein disclosed examples.

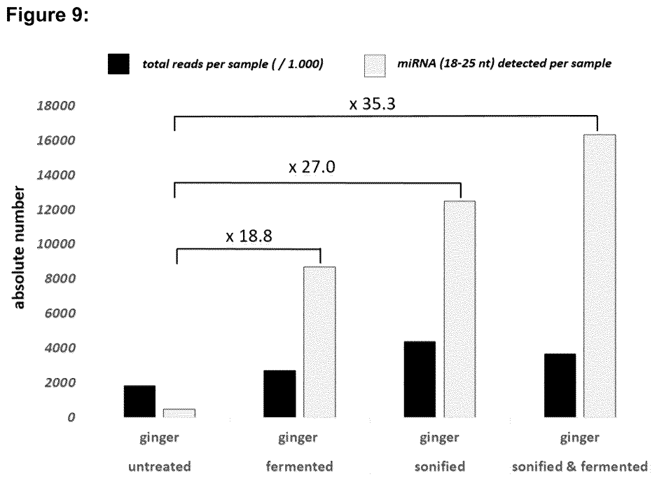

With respect to an enhancement of the quality, as reported herein, e.g., in Example 8, it was surprisingly and advantageously found that upon an acoustic stimulation by ultrasound, optionally succeeded by a fermentation, of non-mammalian cells, i.e., in the present exemplified case, ginger rhizome cells, (i) the overall amount of short RNAs in the size range of 18-25 nts typical for microRNAs (miRNAs) (see, e.g., Ying S Y, Chang D C, Lin S L. The microRNA (miRNA): overview of the RNA genes that modulate gene function. Mol Biotechnol. 2008 March; 38(3):257-68) is increased, and (ii) the ratio of individual known miRNAs is altered (see Example 8: Tables 2 and 3, FIG. 9). Given that in the herein disclosed experiments using extracts from lemon pulp, similar effects with respect to an enhancement of the quantity of produced EVs was observed in case of either of the herein contemplated stimulation techniques, i.e., acoustic stimulation or electromagnetic stimulation, it is at least plausible that similar to an acoustic stimulation (as demonstrated in Example 8), also an electromagnetic stimulation will advantageously provide an improvement of the obtained EVs in terms of the quantity and composition of the therein comprised miRNAs (including known and, as presumed based on their sizes, putative miRNAs).

The term “microRNAs” or “miRNAs”, as used herein, refers to group of small non-coding RNA molecules that are approximately 18-25 nucleotides (nts) in length and regulate the expression of multiple target genes through sequence-specific hybridization to the 3′ untranslated region (UTR) of messenger RNAs (mRNAs). MiRNAs function either by translational inhibition or by causing direct degradation of their target mRNAs. miRNAs resemble the small interfering RNAs (siRNAs) of the RNA interference (RNAi) pathway, except miRNAs derive from regions of RNA transcripts that fold back on themselves to form short hairpins, whereas siRNAs derive from longer regions of double-stranded RNA. The major difference between siRNAs and miRNAs is that the former typically inhibit the expression of one specific target mRNA, while the latter regulate the expression of multiple mRNAs (see, e.g., review articles by Shang R et al., microRNAs in action: biogenesis, function and regulation. Nat Rev Genet. 2023; or Lam J K et al. siRNA Versus miRNA as Therapeutics for Gene Silencing. Mol Ther Nucleic Acids. 2015; 4(9):e252). miRNAs do not require perfect complementarity for target recognition, so a single miRNA is responsible for the regulation of multiple messenger RNAs. The “seed sequence”, “seed region”, or just “seed” is defined as a continuous stretch of 6 to 8 nucleotides within the first 1-10 nucleotides starting at the 5′-end and counting toward the 3′-end of a naturally-occurring mature miRNA, such as one selected from those listed in miRBase (http://www.mirbase.org/; Kozomara A et al., miRBase: from microRNA sequences to function. Nucleic Acids Res. 2019; 47(D1):D155-D162.), and refers to the portion of the miRNA which is essential for the binding of the miRNA to the mRNA. The seed sequence thus typically determines the target mRNA sequence to which the miRNA can bind and provide gene regulation. As such, multiple naturally occurring miRNAs can share a seed sequence, or share substantial homology in the seed sequences, and these miRNAs are members of the same miRNA family. There are several prediction tools publicly available which may be utilized for the sake of predicting miRNA targets in selected organisms. For example, the online tool TargetScanHuman (v8.0) (Agarwal V, Bell G W, Nam J W, Bartel D P. Predicting effective microRNA target sites in mammalian mRNAs. Elife. 2015; 4:e05005; McGeary S E et al. Science. 2019; 366(6472):eaav1741; Lewis B P et al., Cell. 2005; 120(1):15-20) predicts biological targets of miRNAs by searching for the presence of conserved 8mer, 7mer, and 6mer sites that match the seed region of each miRNA. Further predication tools are referred to and discussed in Peterson S M et al., Common features of microRNA target prediction tools. Front Genet. 2014; 5:23. miRNAs play integral roles in several biological processes, including immune modulation, metabolic control, neuronal development, cell cycle, muscle differentiation, and stem cell differentiation. Most miRNAs are conserved across multiple animal species, indicating the evolutionary importance of these molecules as modulators of critical biological pathways and processes. Publicly available databases include, for example, the miRBase, which, in its latest release of 2022 (miRBase v22), contains miRNA sequences from 271 organisms, including 48 860 miRNAs (Kozomara A et al., miRBase: from microRNA sequences to function. Nucleic Acids Res. 2019; 47(D1):D155-D162). Another valuable source is provided by the experimentally validated microRNA-target interactions database miRTarBase (Huang H Y et al., Nucleic Acids Res. 2022; 50(D1):D222-D230; https://mirtarbase.cuhk.edu.cn), which was also employed herein in Example 8 for identifying putative targets of the detected miRNAs from ginger.

In view of the presumed fact that to date the majority of miRNAs, in particular, in non-mammalian species, are yet unknown or at least not experimentally confirmed, the reference herein to “RNA molecules having a size of between 18-25 nts” is intended as an umbrella term to broadly encompass both, known miRNAs and putative miRNAs (i.e., RNA molecules which fall within the typically expected size range of miRNAs, and which are hence suspected, predicted or otherwise suggested to function as miRNAs). Thus, it is understood that any reference herein to an “miRNA” may alternatively be expressed by reference to an “RNA of 18-25 nts length”. Similarly, any reference herein to an miRNA comprising a ‘seed sequence’ (e.g., of a certain nucleotide sequence defined by a SEQ ID NO) may alternatively be expressed by reference to an “RNA of 18-25 nts length comprising a continuous stretch of 6-8 nts (or comprising said specific nucleotide sequence as defined by said SEQ ID NO) within the first 1-10 nts from the 5′-end.

Accordingly, in alternative preferred or even more preferred embodiments of the method according to the first or second aspects of the invention, the amount (i.e., quantity) of RNA molecules having a size in the range of between 18-25 nts and being comprised in, or isolated from, the EVs is increased by at least, with increasing preference, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, or at least 35-fold as compared to the amount of RNA molecules of said size range comprised in, or isolated from, EVs from corresponding cells which have not been subjected to a treatment according to the method according to the first and/or second aspect of the invention.

In preferred embodiments of the latter embodiments, the amount of said RNA molecules is determined/assessed by next-generation sequencing (NGS), more preferably by NGS after isolation of said RNA molecules, wherein preferably said isolation is or has been performed by using a miRNA isolation kit, preferably a column-based miRNA isolation kit; more preferably using the miRNeasy® kit from Qiagen.

In alternative preferred or even more preferred embodiments of the latter embodiments, said RNA molecules are (known and/or putative) micro-RNA (miRNA) molecules.

The skilled person will be aware that for conducting a respective quantification of miRNAs by NGS, the miRNAs need first to be isolated from the EVs before being subjected to NGS. Various methods, including commercially available kits (such as the column based Ambion PureLink™ miRNA Isolation kit from Invitrogen, or the miRNeasy® kit from Qiagen etc.). Various methods for miRNA quantification, including those by NGS are well-known and routinely employed in the art; see, for example, review by Liu J et al., Next generation sequencing for profiling expression of miRNAs: technical progress and applications in drug development. J Biomed Sci Eng. 2011; 4(10):666-676; Hu Y et al. Next-Generation Sequencing for MicroRNA Expression Profile. Methods Mol Biol. 2017:1617:169-177.

Generally, the term “frequency”, as used herein in connection with the definition of the parameters of the acoustic or electromagnetic stimulation, may interchangeably also be referred as “working frequency” and/or “base frequency”.

In further preferred embodiments of the method according to the first aspect of the invention, the non-mammalian cells comprise, consist essentially of, or consist of algae cells, plant cells and/or at least one microorganism.

In some embodiments of the latter embodiments, the non-mammalian cells comprise, consist essentially of, or consist of:
(i) algae cells;
(ii) plant cells and/or at least one microorganism;
(iii) algae cells and at least one microorganism;
(iv) algae cells and plant cells; or
(v) algae cells, plant cells, and at least one microorganism.

In further preferred embodiments, the plant cells comprise, consist essentially of, or consist of cells from one or more fruits, vegetables, legumes, grains, seeds, shoots, sprouts, nuts, leaves, buds, flowers, roots, rhizomes, stolons, tubers, bark and any other plant parts, or any fraction or combination thereof; and/or the at least one microorganism is selected from the group consisting of microbiome-associated bacteria, fungi (i.e., yeasts, molds and/or mushrooms) or any combination thereof.

The term “microbiome-associated bacteria”, as used herein, refers preferably to bacteria which are part of the microbiome of healthy human or animal subjects. In more preferred embodiments, the at least one microorganism is selected from “probiotic bacteria”.

The term “probiotic bacteria” (or just “probiotics”), as used herein, denotes bacteria that, when consumed or administered, can provide health benefits to the host, generally by improving or restoring the gut microbiota. Probiotics have been found to contribute to food digestion, modulate the intestinal microbial communities, suppress growth of pathogens, and enhance host immunity. Recent studies have demonstrated that the beneficial health promoting effects of probiotic bacteria are also attributable to substances (also commonly referred to a “prebiotics”) synthesized by these bacteria and in particular to EVs generated by these bacteria and comprising or carrying such substances (see, e.g., Macia et al., 2019; Rodovalho et al., 2020).

Exemplary and particularly preferred representatives of known probiotic bacteria, of which each may be suitably employed for the methods of the herein disclosed invention include, without limitation, the so-called “lactic acid bacteria” (LAB), which include, inter alia, members of the genera Lactobacillus (e.g., Lactobacillus acidophilus, Lactobacillus casei, Lactobacillus fermentum, Lactobacillus rhamnosu, Lactobacillus delbrueckii, Lactobacillus delbrueckii subsp. bulgaricus, Limosilactobacillus reuteri), Leuconostoc, Pediococcus, Lactococcus, Streptococcus, Aerococcus, Carnobacterium, Escherichia (e.g., Escherichia coli), Enterococcus (e.g., Enterococcus faecium), Oenococcus, Sporolactobacillus, Tetragenococcus, Vagococcus and Weissella. Further known probiotic bacteria include members of the genera Bifidobacterium (e.g., Bifidobacteria bifidum, Bifidobacterium infantis, and Bifidobacteria longum) and Propionibacterium (e.g., Propionibacterium freudenreichii).

In other preferred embodiments, the at least one microorganism is selected from the group of cyanobacteria, preferably Spirulina, more preferably Spirulina platensis.

As used herein, the term “cyanobacteria”, also called “Cyanobacteriota” or “Cyanophyta”, refers to prokaryotic organisms formerly classified as the blue-green algae. Cyanobacteria are a large and diverse group of photosynthetic bacteria which comprise the largest subgroup of Gram-negative bacteria. Cyanobacteria were classified as algae for many years due to their ability to perform oxygen-evolving photosynthesis. The term “Spirulina”, also known as “Arthrospira”, refers to a genus of cyanobacteria and includes the species A. platensis (also known as Spirulina platensis), A. fusiformis, and A. maxim. Due to its high content of, e.g., proteins, polyunsaturated fatty acids, phycobiliproteins, carotenoids, polysaccharides, vitamins, and minerals, and because it can be cultivated easily, Spirulina has long been valued and is widely utilized as a “health food”. The capacity of Spirulina as a functional food for the treatment of disorders like diabetes, cancer, cardiovascular disorders (CVDs), COVID-19, neuroinflammatory conditions and gut dysbiosis, has been acknowledged, for example, in Hirahashi T et al., Int Immunopharmacol. 2002 March; 2(4):423-34; Gómez-Zorita S et al., Int J Mol Sci. 2019 Dec. 19; 21(1):41; Ahmad A M R et al., Cell Mol Biol (Noisy-le-grand). 2023 Jan. 31; 69(1):137-144. The technology of the invention can also be utilized for enhancing the production of EVs from cyanobacteria, such as Spirulina cells, and the so obtained EVs will bear great potential for therapeutic and otherwise health-promoting applications.

In preferred embodiments, the plant cells comprise, consist essentially of, or consist of cells from one or more plants, or specific portions thereof, known to comprise pharmaceutically active and/or health-promoting secondary metabolites or other substances, such as polyphenols, including phenolic acids, phenylpropanoids and flavonoids, terpenes (also referred to as terpenoids), alkaloids, tocopherols (vitamin E), lignins, and/or tannins. Other pharmaceutically active and/or health-promoting substances include amino acids, (poly) peptides, enzymes and/or growth factors.

Particularly preferred examples of plant cells include plant cells from, without limitation, chili peppers, ginger, turmeric (Curcuma), cardamom, cinnamon, black pepper, clove, nutmeg, citrus fruits, grapes, dates, figs, goji berry, tamarind, anise (Pimpinella anisum), staranise (Illicium verum), fennel, garlic, Vitex agnus-castus, Withania somnifera (also commonly known as ashwagandha or winter cherry), beans, ginseng, Ginkgo biloba, maca, pollen and/or Gynostemma pentaphyllum (also known as jiaogulan).

In even more preferred embodiments, the plant cells comprise, consist essentially of, or consist of cells from fruits, preferably citrus fruits, more preferably from lemon fruits.

In alternative preferred embodiments, the plant cells comprise, consist essentially of, or consist of cells (preferably rhizome cells) from one or more members of the family Zingiberaceae, said members preferably being selected from the group consisting of ginger (Zingiber officinale), galangal (also known as Thai ginger (e.g., Alpinia galanga)), melegueta pepper (Aframomum melegueta), myoga (Zingiber mioga), korarima (Aframomum corrorima), turmeric (Curcuma), and cardamom (Amomum or Elettaria).

In preferred embodiments of the latter embodiments, the plant cells comprise, consist essentially of, or consist of cells from turmeric and/or ginger; preferably of turmeric rhizome cells and/or ginger rhizome cells, most preferably ginger rhizome cells.

In alternative preferred embodiments, the plant cells comprise, consist essentially of, or consist of cells from grapes.

The term “algae”, as used herein, refers in line with its common meaning in the art to a diverse group of photosynthetic, simple nonflowering plant-like eukaryotic organisms ranging in size from single-celled diatoms (microalgae) to giant multicellular forms such as kelp or seaweed (macroalgae). It is a polyphyletic grouping that includes species from multiple distinct clades. Included organisms range from unicellular microalgae, such as Chlorella, Prototheca and the diatoms, to multicellular forms, such as the giant kelp, a large brown alga which may grow up to 50 meters in length. Most are aquatic and lack many of the distinct cell and tissue types, such as stomata, xylem and phloem that are found in land plants. The largest and most complex marine algae are called seaweeds, while the most complex freshwater forms are the Charophyta, a division of green algae which includes, for example, Spirogyra and stoneworts. Algae that are carried by water are plankton, specifically phytoplankton. Algae have been shown to exert a variety of health effects, including antiviral, antibacterial, antioxidant, anti-inflammatory, immune enhancing, probiotic, cholesterol-lowering and even anti-cancer effects (see Shan B E et al., Int J Immunopharmacol. 1999 January; 21(1):59-70; Xin Z et al., Int J Nanomedicine. 2023 Sep. 14; 18:5243-5264). Accumulating evidence has revealed for many algae to comprise certain constituents, including metabolites and miRNAs, which bear great therapeutic potential. For example, phlorotannins and fucoidan from the brown algae Fucus vesiculosus were reported to have anti-tumor activity (Catarino M D et al., Int J Mol Sci. 2021 Jul. 16; 22(14):7604; Reyes M E et al., Mar Drugs. 2020; 18(5):232). Many of these constituents are also comprised in the EVs produced by algae. For a review on EVs from algae and their therapeutic potential see, e.g., Bayat F et al., Algal Cells-Derived Extracellular Vesicles: A Review With Special Emphasis on Their Antimicrobial Effects. Front Microbiol. 2021; 12:785716). The present technology can also be utilized for enhancing the production of EVs from algae cells, in terms of both, their quantity and quality, and the so obtained EVs will thus bear great potential for the various therapeutic and otherwise health-promoting applications as contemplated herein below.

In preferred embodiments, the algae cells are selected from green algae (Chlorophyta), brown algae (Phaeophyta) and/or diatoms (Bacillariophyta).

In other or even more preferred embodiments, the non-mammalian cells comprise, consist essentially of, or consist of plant cells and at least one microorganism selected from the genus Lactobacillus.

In other or even more preferred embodiments, the non-mammalian cells are comprised in a maintenance medium or culture medium, preferably a maintenance or culture medium comprising, consisting essentially of, or consisting of an aqueous solution or water. Preferably, the maintenance or culture medium or the aqueous suspension comprises at least 40 vol % water (preferably extracellular water) with respect to the total weight or the total volume.

It is presumed by the inventors that the non-mammalian cells, may it be through active or passive import processes or both, and, in particular, as a further advantageous effect of the acoustic and/or electromagnetic stimulation, will be able to take up substances from the extracellular milieu, e.g., the surrounding maintenance or culture medium, and that the so generated EVs will consequently also comprise such substances. It is hence also expressly contemplated herein to exploit these mechanisms to incorporate biologically and/or pharmaceutically active ingredients, in particular substances known to provide a health-promoting effect and/or a treatment effect for certain nutritional deficiencies or specific pathological conditions, into the EVs generated by the method of the invention.

Accordingly, in preferred embodiments, it is contemplated herein that the maintenance or culture medium, or the aqueous suspension, further comprises one or more biologically or pharmaceutically active substances, preferably selected from vitamins, preferably water-soluble vitamins (such as vitamin C and/or B-complex vitamins, such as vitamin B6, vitamin B12, and folate (also known as folic acid or vitamin B9)), trace elements (such as iodine, iron, selenium, and/or zinc), secondary plant metabolites (examples of which are also described herein above), methylsulfonylmethane (MSM), and/or amino acids, (poly) peptides or derivatives thereof (e.g., carnitine, carnosine).

In certain preferred embodiments, the method further comprises, after the stimulation in step (a), yet before the optional collecting in step (b), an incubation (i.e., a post-stimulation incubation). Although an enhanced production of EVs from the cells was already observed from the time point immediately after the onset of the applied acoustic or electromagnetic stimulation, it was advantageously found by the present inventors that this effect was even more pronounced when the cells, after the stimulation, were allowed to rest for a certain amount of time, e.g., about 24 hours at 6° C., prior to the collection of the EVs.

Thus, in particular preferred embodiments, said incubation is conducted:
(i) at a temperature in the range of between, with increasing preference, 1° C. and 42° C., 1° C. and 37° C., 1° C. and 35° C., 1° C. and 30° C., 1° and 25°, 1° C. and 20° C., 1° C. and 15° C., 1° C. and 10° C., 2° C. and 9° C., 3° C. and 8° C., 4° C. and 7° C., most preferably at 6° C.; and/or
(ii) for a time period in the range of between, with increasing preference, 1 hour to 72 hours, 2 hours to 60 hours, 6 hours to 48 hours, 12 hours to 36 hours, 18 hours to 30 hours, 20 hours to 28 hours, most preferably 24 hours.

In alternative preferred embodiments, said incubation is conducted:
(i) at a temperature in the range of between, with increasing preference, 1° C. and 42° C., 1° C. and 37° C., 1° C. and 35° C., 1° C. and 30° C., 1° and 25°, 1° C. and 20° C., 1° C. and 15° C., 1° C. and 10° C., 2° C. and 9° C., 3° C. and 8° C., 4° C. and 7° C., most preferably at 6° C.; and/or
(ii) for a time period of, with increasing preference, at least 0.5 hours, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, and most preferably for a time period of at least 24 hours.

In further preferred embodiments of the method according to the first aspect of the invention, the collecting in step (b) is conducted by:
(i) centrifugation, thereby obtaining a centrifugation supernatant comprising the EVs;
(ii) precipitation of the EVs from the centrifugation supernatant by contacting the centrifugation supernatant with a water excluding polymer;
(iii) pelleting the precipitated EVs by centrifugation and disposal of the centrifugation supernatant; and optionally
(iv) resuspending the pelleted EVs in an aqueous buffer.

In particular preferred embodiments of the latter embodiments, step (i) is conducted by a differential centrifugation based on two or more consecutive centrifugation steps, wherein the centrifugation force is progressively increased by each further centrifugation step relative to the preceding centrifugation step;

and wherein preferably:
the water excluding polymer in step (ii) is selected from polyethylene glycol (PEG), dextran, dextran sulfate, dextran acetate, polyvinyl alcohol, polyvinyl acetate and polyvinyl sulfate, or any combination thereof; and/or
the precipitation in step (ii) is conducted by addition of the water excluding polymer to the centrifugation supernatant, followed by an incubation for, with increasing preference, at least 6 hours, at least 8 hours, at least 10 hours, at least 12 hours, or at least 14 hours and at a temperature below 25° C., more preferably below 15° C., even more preferably below 10° C., and most preferably at 4° C.; and/or
the aqueous buffer in step (iv) is an essentially physiological aqueous buffer, more preferably phosphate buffered saline (PBS) pH 7.4.

The term “physiological aqueous buffer”, as used herein, refers to an aqueous solution having a total salt concentration (and consequently osmolarity) and pH which match that of a physiological liquid, such as blood. Examples of physiological aqueous buffers include, but are not limited to, phosphate-buffered saline (PBS) pH 7.4, Hanks' balanced salt solution, ringer's solution and the like.

The term “essentially physiological”, as used herein with respect to buffer solutions means that the total salt concentration and/or the pH can deviate by a maximum of +/−25%, preferably by a maximum of +/−20%, more preferably by a maximum of +/−10%, more preferably by a maximum of +/−5%, from the corresponding physiological condition.

In even more preferred embodiments of the latter embodiments, the differential centrifugation in step (i) is conducted at 4° C. and by the three consecutive centrifugation steps in the order: 300×g for 10 min, 2,000×g for 30 min and 10,000×g for 30 min.

In preferred embodiments of the method according to the first aspect of the invention, the acoustic stimulation in step (a) is conducted by applying a continuous or pulsed, preferably a pulsed, ultrasound:
(i) at a frequency in the range of between, with increasing preference, 20 KHz and 10 MHz, 25 kHz and 9 MHz, 50 KHz and 8 MHz, 100 KHz and 7 MHz, 250 kHz and 6 MHz, 500 KHz and 4 MHz, 1000 kHz and 2 MHz, 0.2 MHz and 1.8 MHz, 0.4 MHz and 1.6 MHz, 0.6 MHz and 1.4 MHz, 0.8 MHz and 1.2 MHz, most preferably at a frequency of about 1 MHz;
(ii) with an intensity in the range of between, with increasing preference, 0.5 mW/cm² and 3 W/cm², 10 mW/cm² and 750 mW/cm², 10 mW/cm² and 750 mW/cm², 50 mW/cm² and 600 mW/cm², 100 mW/cm² and 500 mW/cm², 200 mW/cm² and 360 mW/cm², 230 mW/cm² and 330 mW/cm², 260 mW/cm² and 300 mW/cm², most preferably of about 280 mW/cm²;
(iii) with a duty cycle in the range of between, with increasing preference, 1% and 100%, 2% and 90%, 3% and 80%, 4% and 70%, 5% and 60%, 6% and 50%, 7% and 40%, 8% and 35%, 10% and 30%, 15% and 25%, most preferably about 20%;
(iv) with a pulse duration in the range of between, with increasing preference, 1 s and 20 min, 2 s and 10 min, 3 s and 5 min, 5 s and 60 s, 10 s and 50 s, 20 s and 40 s, most preferably about 30 s;
(v) for a total time period in the range of between, with increasing preference, 5 seconds to 12 hours, 10 seconds to 6 hours, 30 seconds to 3 hours, 1 minute to 1.5 hours, 2 minutes to 1 hour, 3 minutes to 40 minutes, 4 minutes to 30 minutes, 5 minutes to 25 minutes, most preferably about 15 minutes; and/or
(vi) at a temperature in the range of between, with increasing preference, 1° C. and 42° C., 1° C. and 37° C., 1° C. and 35° C., 1° C. and 30° C., 1° C. and 25° C., 1° C. and 20° C., 1° C. and 15° C., 1° C. and 10° C., 2° C. and 6° C., 3° C. and 5° C., and most preferably at about 4° C.

As explained herein above, in the context of the ultrasound stimulation, a duty cycle refers to the percentage of time during which the ultrasound signal is delivered (“on”) relative to the total time of the ultrasound treatment. In the herein disclosed examples (e.g., Example 2), the total period of the applied ultrasound stimulation was 15 min with a duty cycle set to “20%”, accordingly meaning that the ultrasound was “on” for a period of 3 min in total. In the specific setting applied, this period of 3 min total “on”-time was split into six intervals (30 s each) and distributed equally over the 15 mins of total time (i.e., as 5×(30 s ultrasound+144 s pause) followed by 30 s ultrasound=15 min).

In particularly preferred embodiments of the latter embodiments, in the instance of the ultrasound being pulsed, the ultrasound is applied at a frequency of 1 MHz with an intensity of about 280 mW/cm² for a total period of about 15 min with a 20% duty cycle, with a pulse duration of about 30 s, and at a temperature of about 4° C.

In further preferred embodiments of the method according to the first aspect of the invention, in the instance of the stimulation in step (a) being conducted by applying ultrasound and no electromagnetic stimulation, the concentration of the EVs obtained in step (b) is:
(i) increased by, with increasing preference, at least 100%, 200%, 300%, 350%, 360%, 370%, 380%, 390%, 400%, 410%, most preferably by at least 415%, as compared to the concentration of EVs obtainable from corresponding cells in the absence of a respective stimulation by ultrasound; and/or
(ii) greater than 1×10⁹/ml, preferably greater than 1×10¹⁰/ml, and more preferably about 1×10¹¹/ml;
preferably as determinable by nanoparticle tracking analysis (NTA).

In preferred embodiments of the method according to the first aspect of the invention, in the instance of the stimulation in step (a) being conducted by applying ultrasound and no electromagnetic stimulation, the mean size of the EVs obtained in step (b) is:
(i) reduced by, with increasing preference, at least 10%, 11%, 12%, 13%, 14%, 15%, 16%, 17%, 18%, 19%, 20%, 21%, 22%, 23%, 24%, most preferably by at least 25% as compared to the mean size of EVs obtainable from corresponding cells in the absence of a respective stimulation by ultrasound; and/or
(ii) below, with increasing preference, 210 nm, 205 nm, 200 nm, 195 nm, 190 nm, 185 nm, 180 nm, 175 nm, 170 nm, most preferably below 165 nm; and/or
(iii) about 162.6±85 nm;
preferably as determinable by nanoparticle tracking analysis (NTA).

In further preferred embodiments of the method according to the first aspect of the invention, the electromagnetic stimulation in step (a) is conducted by applying a continuous or pulsed, preferably a pulsed, electromagnetic radiation:
(i) at a frequency in the range of between, with increasing preference, 20 KHz and 500 kHz, 30 KHz and 450 kHz, 80 KHz and 400 kHz, 130 kHz and 350 kHz, 180 KHz and 300 kHz, 200 kHz and 280 kHz, 220 kHz and 260 kHz, most preferably about 240 kHz;
(ii) with an energy output in the range of between, with increasing preference, 54 joule per pulse and 96 joule per pulse, 55 joule per pulse and 90 joule per pulse, 55 joule per pulse and 80 joule per pulse, 55 joule per pulse and 70 joule per pulse, 55 joule per pulse and 65 joule per pulse, most preferably about 60 joule per pulse;
(iii) with a pulse duration in the range of between, with increasing preference, 10 μs and 120 μs, 20 μs and 100 μs, 30 μs and 80 μs, 40 μs and 60 μs, most preferably about 50 μs;
(iv) with a pulse repetition rate in the range of between, with increasing preference, 0.1 Hz and 20 Hz, 0.5 Hz and 15 Hz, 0.6 Hz and 10 Hz, 0.8 Hz and 5 Hz, 1 Hz and 3 Hz, most preferably about 2 Hz;
(v) for a total time period in the range of between, with increasing preference, 5 seconds to 12 hours, 10 seconds to 6 hours, 30 seconds to 3 hours, 1 minute to 1.5 hours, 2 minutes to 1 hour, 3 minutes to 40 minutes, 4 minutes to 30 minutes, 5 minutes to 25 minutes, most preferably about 15 minutes; and/or
(vi) at a temperature in the range of between, with increasing preference, 1° C. and 42° C., 1° C. and 37° C., 1° C. and 35° C., 1° C. and 30° C., 1° and 25°, 1° C. and 20° C., 1° C. and 15° C., 1° C. and 10° C., 2° C. and 6° C., 3° C. and 5° C., and most preferably at about 4° C.

In particularly preferred embodiments of the latter embodiments, in the instance of the electromagnetic radiation being pulsed, the electromagnetic radiation is most preferably applied at a frequency of about 240 kHz with a pulse duration of about 50 μs, with a pulse repetition rate of about 2 Hz, and an energy output of about 60 joule per pulse.

In alternative preferred embodiments, in the instance of the electromagnetic radiation being continuous, the electromagnetic radiation is applied:
(i) at a frequency in the range of between, with increasing preference, 20 KHz and 500 kHz, 30 KHz and 450 kHz, 80 KHz and 400 kHz, 130 kHz and 350 kHz, 180 KHz and 300 kHz, 200 kHz and 280 kHz, 220 KHz and 260 kHz, most preferably about 240 kHz;
(ii) with an energy output in the range of between, with increasing preference, 0.1 MW/m² and 2 MW/m², 0.5 MW/m² to 1.5 MW/m², 1.0 MW/m² and 1.4 MW/m², most preferably about 1.2 MW/m²;
(iii) for a total time period in the range of between, with increasing preference, 5 seconds to 12 hours, 10 seconds to 6 hours, 30 seconds to 3 hours, 1 minute to 1.5 hours, 2 minutes to 1 hour, 3 minutes to 40 minutes, 4 minutes to 30 minutes, 5 minutes to 25 minutes, most preferably about 15 minutes; and/or
(iv) at a temperature in the range of between, with increasing preference, 1° C. and 42° C., 1° C. and 37° C., 1° C. and 35° C., 1° C. and 30° C., 1° and 25°, 1° C. and 20° C., 1° C. and 15° C., 1° C. and 10° C., 2° C. and 6° C., 3° C. and 5° C., and most preferably at about 4° C.

The skilled person will be aware that a continuous electromagnetic radiation may, dependent on the energy output and duration of the radiation applied, give rise to more heating effects than an electromagnetic radiation which is applied as pulsed (i.e., interrupted by pauses during which no energy is applied), and that these heating effects may negatively interfere with the survival of the non-mammalian cells and/or their capacity to produce EVs. Being aware of these phenomena, the skilled person, when using a continuous electromagnetic stimulation, will be able to suitably adjust the overall energy output and duration of the electromagnetic radiation to circumvent any excessive heating.

Thus, in other exemplary embodiments of the method, where a continuous electromagnetic radiation is being applied, the electromagnetic radiation is applied with an energy output which does not exceed, with increasing preference, 1.5 MW/m², 1.4 MW/m², 1.3 MW/m², 1.2 MW/m², 1.1 MW/m², 1.0 MW/m², 0.9 MW/m², 0.8 MW/m², 0.7 MW/m², 0.6 MW/m², 0.5 MW/m², 0.4 MW/m², 0.3 MW/m², 0.2 MW/m², 0.1 MW/m², 90 kW/m², or 80 kW/m².

In further preferred embodiments of the method according to the first aspect of the invention, in the instance of the stimulation in step (a) being conducted by applying an electromagnetic stimulation and no ultrasound, the concentration of the EVs obtained in step (b) is
(i) increased by, with increasing preference, at least 100%, 200%, 300%, 310%, 320%, 330%, 340%, 350%, most preferably by at least 355%, as compared to the concentration of EVs obtainable from corresponding cells in the absence of a respective stimulation by ultrasound; and/or
(ii) greater than 1×10⁹/ml, preferably greater than 1×10¹⁰/ml, and more preferably about 1×10¹¹/ml;
preferably as determinable by nanoparticle tracking analysis (NTA).

In further preferred embodiments of the method according to the first aspect of the invention, in the instance of the stimulation in step (a) being conducted by an electromagnetic stimulation and no ultrasound, the mean size of the EVs obtained in step (b) is
(i) reduced by, with increasing preference, at least 1%, 2%, 3%, 4%, 5%, most preferably by at least 6% as compared to the mean size of EVs obtainable from corresponding cells in the absence of a respective stimulation by electromagnetic radiation; and/or
(ii) below 210 nm, with increasing preference, below 205 nm, 204 nm, most preferably below 203 nm; and/or
(iii) about 202.8+99 nm;
preferably as determinable by nanoparticle tracking analysis (NTA).

In further preferred embodiments, the non-mammalian cells, prior to step (a), are or have been subjected to a fermentation.

The term “fermentation” or “fermenting”, as interchangeably used herein, refers broadly and in line with its common meaning in biology and biotechnology, to a metabolic process that produces chemical changes in organic substrates through the action of enzymes. This may be established either intracellularly, i.e., through the action of enzymes comprised in cells, in the present case the non-mammalian cells (such as microbial cells (e.g., bacterial and/or fungal (e.g., yeast) cells) and/or plant cells), which carry out the fermentation within the framework of their enzyme-catalyzed metabolism or extracellularly, i.e., through the action of enzymes which have been secreted by these cells or added otherwise to the surrounding maintenance or culture medium.

Thus, the feature that the non-mammalian cells are “subjected to a fermentation”, as used herein, means broadly that these cells are subjected to an incubation under aerobic or anaerobic atmospheric conditions, preferably under (at least substantially) anaerobic atmospheric conditions, for a sufficient amount of time that a fermentation can occur. As such, according to the broadest definition, the post-stimulation incubation period as referred to herein above may also be regarded as fermentation. However, in preferred embodiments, the post-stimulation incubation is conducted under aerobic atmospheric conditions, and the fermentation is conducted at substantially anaerobic atmospheric conditions.

In a more preferred embodiment, the terms “fermentation” or “fermenting”, as used herein, refer more narrowly, and in line with their common meaning in food processing, to the conversion of carbohydrates to alcohol or organic acids using microorganisms, typically selected from fungi (preferably yeasts) and bacteria, under (at least substantially) anaerobic (i.e., oxygen-free) conditions. It is understood that in such embodiments, the non-mammalian cells comprise at least one microorganism, preferably selected from fungi (preferably yeasts) and bacteria, more preferably from lactic acid bacteria (LAB), and the fermentation is conducted under anaerobic (i.e., oxygen-free) or at least substantially anaerobic conditions. The term “substantially anaerobic atmospheric condition”, as used herein in reference to a fermentation condition, is intended to mean that an atmosphere of less than about 1% oxygen is maintained during the fermentation, and alternatively means that the dissolved oxygen concentration is 0.1 ppm or less, or even 0.01 ppm or less. The skilled person is aware that for providing such conditions, the oxygen supply can be restricted, for example, by reducing the aeration, limiting the stirring, and/or introducing a gas which has a reduced partial pressure of oxygen by mixing in an inert gas, such as nitrogen or carbon dioxide. The anaerobic atmosphere can be made completely anaerobic by removing the oxygen entirely. To completely remove the oxygen, aeration can be stopped, or only an inert gas can be introduced, or the like. Suitable small and large-scale bioreactors for conducting anaerobic fermentations are commercially available and routinely used, e.g., in the food and beverage industries.

The skilled person will understand, based on the type of non-mammalian cells that are to be employed in the method of the invention, that either aerobic or anaerobic conditions may be more suitable for conducting a fermentation. For example, several microorganisms, such as lactic acid bacteria (LAB) as further described hereafter, which are prominently used in fermentation processes in the food industry, typically require (at least substantially) anaerobic conditions for their growth and survival (see, for example, the review articles by Swain et al., 2014; Di Cagno et al., 2013).

In the past, the beneficial effects of fermented foods on health were unknown, and so fermentation was primarily used to preserve foods, enhance shelf life, and improve flavor. Fermented foods became an important part of the diet in many cultures, and over time fermentation has been associated with many health benefits. Because of this, the fermentation process and the resulting fermented products have attracted scientific interest. Microorganisms contributing to the fermentation process have recently been associated with many health benefits, and so these microorganisms have become another focus of attention. Bacteria of the order Lactobacillies, also commonly referred to as lactic acid bacteria (LAB), have been some of the most studied microorganisms. Lactobacillies are an order of gram-positive, low-GC, acid-tolerant, generally non-sporulating, non-respiring, either rod-shaped (bacilli) or spherical (cocci) bacteria that share common metabolic and physiological characteristics. These bacteria, usually found in decomposing plants and milk products, produce lactic acid as the major metabolic end product of carbohydrate fermentation, giving them the common name lactic acid bacteria (LAB). Production of lactic acid has linked LAB with food fermentations, as acidification inhibits the growth of spoilage agents. Proteinaceous bacteriocins are produced by several LAB strains and provide an additional hurdle for spoilage and pathogenic microorganisms. Furthermore, lactic acid and other metabolic products contribute to the organoleptic and textural profile of a food item. The industrial importance of the LAB is further evidenced by their generally recognized as safe (GRAS) status, due to their ubiquitous appearance in food and their contribution to the healthy microbiota of animal and human mucosal surfaces. During fermentation, these bacteria synthesize vitamins and minerals, produce biologically and/or pharmaceutically active (poly) peptides with enzymes such as proteinase and peptidase, and remove some non-nutrients. Compounds known as biologically active peptides, which are produced by the bacteria responsible for fermentation, are also well known for their health benefits.

The skilled person will understand that a fermentation can, in principle, already occur in the presence of the non-mammalian cells alone, i.e., without the need of any additional constituents, namely either intracellularly, i.e., through enzymatic conversion of substrates being intracellularly comprised in the non-mammalian cells or taken up from the extracellular milieu by some of the non-mammalian cells upon secretion from other non-mammalian cells, or extracellularly, through enzymes which may be secreted by the non-mammalian cells and consequently act on substrates displayed on, or released from, the non-mammalian cells. However, in more preferred embodiments, the non-mammalian cells are comprised in a medium (i.e., a maintenance or culture medium) comprising one or more additional constituents that may serve as substrate(s) for the fermentation, i.e., for being intracellularly and/or extracellularly enzymatically converted, and which conversion products may then be incorporated into the EVs produced by the non-mammalian cells.

For example, in preferred embodiments, the maintenance medium or culture medium additionally comprises natural raw material from plants selected from the group consisting of fruits, vegetables, legumes, grains, seeds, shoots, sprouts, nuts, leaves, buds, flowers, roots, rhizomes, stolons, tubers, bark and any other plant parts, or any fraction or combination thereof.

It will also be readily appreciated by the skilled person that, dependent on the specific non-mammalian cells which are selected for being employed in the fermentation, it can be productive to adjust the composition of the medium (i.e., the maintenance or culture medium) to promote the fermentation process.

For example, it is common practice in the fermentation of plant raw materials (e.g., vegetables or fruits), in particular, in those fermentations conducted in the presence of microorganism(s) such as LAB (i.e., a so-called lacto-fermentation), to add salt to the raw material or to supplement the maintenance or culture medium with salt (typically sodium chloride, typically 2-4% w/v). This provides several beneficial effects: it helps to draw water including therein dissolved substrates out of the plant material and thus creates a natural brine, full of nutrients for the microorganisms. It also helps to cover the plant raw material and thereby prevents exposure to oxygen, creating an environment most suitable for fermentation. In addition, salt acts as a preservative by favoring the growth/survival of the desired microorganisms (e.g., LAB) over that of any pathogenic microorganisms which may potentially be present as impurities in the raw material, reaction vessel, or atmosphere. In general, the skilled person will understand that the higher the salt concentration, the more the microorganism(s), including any potential pathogenic microorganisms, will be hampered in their growth and that the less salt, the faster the fermentation will proceed while having higher risk for contamination.

Thus, in preferred embodiments, the medium (i.e., the maintenance medium or culture medium) comprises salt, preferably between 1% and 10% (w/v), more preferably 2-4% (w/v).

It is also common practice in fermentation of plant raw material with microorganisms to add one or more carbohydrate substrate(s) to the raw material or to supplement the medium (i.e., the maintenance medium or culture medium) with carbohydrate substrate(s) in order to provide these as a so-called fermentation starter, i.e., as a source of energy for the microorganisms to promote their survival and/or growth and/or metabolic activity.

Thus, in alternative or even further preferred embodiments, the medium (i.e., the maintenance medium or culture medium) additionally comprises one or more carbohydrate substrate(s). Preferably, the carbohydrate substrate is selected from the group consisting of fructose, glucose, galactose, maltose and lactose, or any combination thereof.

In further alternative or even more preferred embodiments, the non-mammalian cells may be isolated from the medium (i.e., the maintenance medium or culture medium) used for the fermentation, or the medium (i.e., a maintenance medium or culture medium) used for the fermentation is replaced or diluted with a different medium (i.e., a maintenance medium or culture medium), prior to being subjected to step (a) of the method according to the first aspect of the invention.

In a second aspect, the invention relates to a method of (or for) fermenting non-mammalian cells, the method comprising:
(A) obtaining non-mammalian cells by the method according to the first aspect of the invention; and
(B) subjecting the cells to a fermentation;
   wherein preferably the fermentation comprises:
   (i) admixing the non-mammalian cells with:
      an aqueous salt solution; and/or
      at least one microorganism selected from the group consisting of microbiome-associated bacteria, yeasts and molds, or any combination thereof; and/or
      a carbohydrate substrate selected from the group consisting of fructose, glucose, galactose, maltose and lactose, or any combination thereof;
      and/or
   (ii) incubating, under substantially anaerobic atmospheric conditions, for a sufficient amount of time to allow the fermentation to proceed;
   wherein more preferably:
   (i-a) the aqueous salt solution:
      comprises at least one salt, preferably sodium chloride, at a total concentration in the range of between, with increasing preference, 0.1% (w/v) and 20% (w/v), 0.25% (w/v) and 15% (w/v), 0.5% (w/v) and 10% (w/v), 0.75% (w/v) and 7.5% (w/v), 1% (w/v) and 5% (w/v), 2% (w/v) and 4% (w/v), most preferably 3% (w/v); and/or
      is added to result in a concentration in the range of between, with increasing preference, 5-95%, 10-90%, 15-85%, 20-80%, 25-75%, 30-70%, 35-65%, 40-60%, most preferably 45-55% of the total volume; and/or
   (i-b) the at least one microorganism is selected from the genus Lactobacillus; and/or
   (i-c) the carbohydrate substrate is added at a concentration in the range of between, with increasing preference, 10 g and 1 kg per L or per kg natural raw material;
(C) optionally, enriching the EVs from the natural raw material as obtained after step (B).

In a third aspect, the invention relates to EVs obtainable or that have been obtained by the method according to the first or second aspect of the invention.

As is evident from the herein disclosed examples, the EVs obtained by the method of the present invention were found to have reduced sizes (i.e., vesicle diameters) as compared to EVs obtained from respective non-mammalian cells not subjected to a corresponding acoustic or electromagnetic stimulation (see FIGS. 7A and 7B). Based on these observations, it is presumed that the EVs obtained by the method of the invention can be distinguished from the prior art EVs, not only based on their reduced sizes, but also on further structural characteristics, such as the particular composition of the constituents of their intravesicular cargo and/or the composition of their surrounding membrane or molecules embedded or anchored therein or displayed thereon. One particular contemplated scenario is, yet without wishing to be bound by any theory, that, in instances where the non-mammalian cells comprise a mixture of cells of different origin, e.g., plant cells and bacterial cells, that an exposure to an acoustic and/or electromagnetic stimulation as conducted in accordance with the method of the invention will, besides providing an enhancement of the production and release of EVs, enhance the permeability of the cell membrane and thereby an exchange of EVs between these different cells to give rise to “hybrid” EVs which comprise/carry cargo of distinct cellular origin.

Moreover, as is also evident from the herein below disclosed examples, the EVs obtained by the method according to the first aspect of the present invention, when applied on target cells (e.g., PBMCs), were unexpectedly and advantageously found to improve the bioenergetic status (i.e., improved mitochondrial function) of these target cells. This advantageous capacity was found to be superior as compared to EVs obtained by conventional routes from the same kind of non-mammalian cells (see Example 7 and FIG. 8). Hence, the EVs generated by the method of the invention can, likely as a direct or indirect effect of the acoustic or electromagnetic stimulation, be distinguished from conventionally produced EVs at least by these improved functional properties.

Furthermore, an analysis of the EVs obtained from ginger rhizome cells by the method according to the first or second aspect of the invention experimentally confirmed that qualitative and quantitative changes occurred with respect to their RNA cargo, in particular their contents of (known and/or putative) miRNAs, as a result of said treatment. A subsequent database search revealed for at least one of the detected miRNAs from the obtained ginger EVs to possess an identical ‘seed sequence’ as two known human miRNAs (hsa-miR-1269a and hsa-miR-1269b; see Example 8, Table 4) and for which a critical implication in the regulation of several important health-promoting physiological pathways is known (experimentally confirmed) or predicted (see Example 8, Table 5). Given the fact that miRNA function is determined by its seed sequence, and in view of the accumulating evidence for cross-kingdom and cross-species functionality of EVs and miRNAs, it is highly likely that at least such miRNAs from non-mammalian cells (such as ginger) having an identical or at least substantially identical seed sequence to human miRNAs of known or predicted function also have the capacity to act as functional paralogs of the latter. In addition, it is at least plausible that certain EV-derived miRNAs for which no human analogous miRNAs exist may comprise a seed sequence complementary to human mRNAs and may thus have the capacity to act as expression regulators of the underlying human genes. Without wishing to be bound by any theory, this may provide a possible rationale for the frequently reported health-promoting effects (such as, inter alia, anti-inflammatory, antioxidant, and anti-tumor benefits) of certain non-mammalian (e.g., microbe- or plant-derived) EVs (Sarasati A et al. Plant-Derived Exosome-like Nanoparticles for Biomedical Applications and Regenerative Therapy. Biomedicines. 2023; 11(4): 1053; Loogozzi M et al. The Potentiality of Plant-Derived Nanovesicles in Human Health—A Comparison with Human Exosomes and Artificial Nanoparticles. Int J Mol Sci. 2022; 23(9): 4919; Zhang B et al. The Potential Role of Gut Microbial-Derived Exosomes in Metabolic-Associated Fatty Liver Disease: Implications for Treatment. Front Immunol. 2022; 13: 893617; Diez-Sainz E et al. Effects of gut microbiota-derived extracellular vesicles on obesity and diabetes and their potential modulation through diet. J Physiol Biochem. 2022; 78(2): 485-499) and may as well underlie, at least in part, the herein identified improvements of mitochondrial function and cellular bioenergetics in human PBMCs upon treatment with EVs from lemon pulp (cf. Example 7).

It was found herein that the EVs obtained from ginger rhizome cells upon being subjected to the method according to the first or second aspect of the invention can be structurally distinguished from EVs from untreated ginger rhizome cells by their miRNA cargo. Although the EVs from all test samples (i.e., independent of the treatment) were found to contain miRNAs of the known (conserved) ginger miRNA families MIR319, MIR159, MIR396, MIR168, MIR156, and MIR164, the EVs obtained by the methods according to the first and second aspects of the present invention were surprisingly found to comprise these miRNAs at increased quantities and with a distinct order of abundance, with miRNAs of the MIR319 and MIR159 families being the most abundant ones among said six known miRNA families (see Example 8, Table 1). The miRNAs of the ginger miRNA family MIR319 have a seed sequence that is identical to the human miRNAs hsa-miR-1269a and hsa-miR-1269b and are therefore presumed to act as functional analogs/paralogs of the latter (see Example 8, Table 4). The latter human miRNAs are known to be positively implicated in the expression regulation of several human genes known to underlie critical health-promoting pathways (see Example 8, Table 5: Reactome pathways). The EVs of the invention, since comprising such miRNAs (esp. those of the MIR319 family) at high abundance, are thus expected to provide a positive health-promoting, therapeutic and/or preventive effect upon being administered to a human subject.

Thus, in preferred embodiments of the EVs according to the third aspect of the invention, the EVs comprise at least one miRNA comprising a seed sequence as defined by SEQ ID NO: 63 (i.e., corresponding to the seed sequence of the ginger miRNA miR319_1; see Tables 1 and 4); and wherein preferably:
said miRNA is comprised in said EVs at a frequency (i.e., with an abundance) which is at least, with increasing preference, 5-fold, 6-fold, 7-fold, 8-fold, 9-fold, 10-fold, 15-fold, 16-fold, 17-fold, 18-fold, or at least 19-fold higher as compared to said miRNA comprised in EVs obtainable or obtained from corresponding ginger cells which have not been subjected to a treatment according to the method according to the first and/or second aspect of the invention; and/or
said at least one miRNA is preferably independently selected from any one of SEQ ID NOs: 31-44 (more preferably from any one of SEQ ID NOs: 31-34 and 36-44).

As shown in FIG. 9, it was furthermore surprisingly found that EVs according to the third aspect of the invention, in particular those from ginger rhizome cells, have an increased content of miRNAs as compared to EVs obtained from corresponding untreated cells.

Thus, in preferred embodiments of the EVs according to the third aspect of the invention, the amount (i.e., quantity) of miRNAs (or RNA molecules having a size in the range of between 18-25 nts) comprised in the EVs is increased (preferably at least, with increasing preference, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, or at least 35-fold higher as compared to the amount of miRNAs (or RNA molecules having a size in the range of between 18-25 nts) comprised in EVs which have not been subjected to a treatment according to the method according to the first and/or second aspect of the invention.

In alternative preferred embodiments of the EVs according to the third aspect of the invention, the amount (i.e., quantity) of RNA molecules having a size in the range of between 18-25 nts being comprised in, or isolated from, the EVs is increased by at least, with increasing preference, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, or at least 35-fold as compared to the amount of RNA molecules of said size range comprised in, or isolated from, EVs from corresponding cells which have not been subjected to a treatment according to the method according to the first and/or second aspect of the invention.

In preferred embodiments of the latter embodiments, the amount of said RNA molecules is determined/assessed by next-generation sequencing (NGS), more preferably by NGS after isolation of said RNA molecules, wherein preferably said isolation is or has been performed by using a miRNA isolation kit, preferably a column-based miRNA isolation kit; more preferably using the miRNeasy® kit from Qiagen. The skilled person will understand that a quantification by sequencing (e.g., NGS) does not provide the absolute number of said RNA molecules which were comprised in a sample, but allows a quantification of the content of said RNA molecules in a test sample relative to a reference sample.

In a fourth aspect, the invention relates to EVs from ginger cells, preferably from ginger rhizome cells, wherein:
said EVs comprise one or more miRNAs corresponding to member(s) of each of the ginger miRNA families MIR319, MIR159, MIR396, MIR168, MIR 156 and MIR164, and wherein the one or more miRNAs corresponding to each of the families MIR319 and MIR159 are comprised at a higher abundance as compared to the one or more miRNAs corresponding to each of the families MIR396, MIR168, MIR156, and MIR164; and/or
said EVs comprise at least one miRNA from each of the following groups (i) to (vi): (i) SEQ ID NOs: 1-13; (ii) SEQ ID NOs: 14-17; (iii) SEQ ID NOs: 18-24; (iv) SEQ ID NOs: 25-30; (v) SEQ ID NOs: 31-44; and (vi) SEQ ID NOs: 45-30, wherein the total amount of the miRNA(s) corresponding to each of the groups (ii) and (v) is higher relative to the total amount of the miRNA(s) corresponding to each of the groups (i), (ii), (iv), and (vi).

In preferred embodiments of the EVs according to the fourth aspect of the invention, the EVs are obtainable or have been obtained by the method according to the first or second aspect of the invention.

In preferred embodiments of the latter embodiments, the EVs comprise at least one miRNA comprising a seed sequence as defined by SEQ ID NO: 63 (i.e., corresponding to the seed sequence of the ginger miRNA miR319_1: see Example 8, Table 4); and wherein preferably:
said EVs are obtainable or have been obtained by the method according to the first or second aspect of the invention, and wherein said miRNA is comprised at a frequency (i.e., with an abundance) which is at least, with increasing preference, 5-fold, 6-fold, 7-fold, 8-fold, 9-fold, 10-fold, 15-fold, 16-fold, 17-fold, 18-fold, or at least 19-fold higher as compared to said miRNA comprised in EVs obtainable or obtained from corresponding ginger cells which have not been subjected to a treatment according to the method according to the first or second aspect of the invention; and/or
said at least one miRNA is independently selected from any one of SEQ ID NOs: 31-44 (more preferably from any one of SEQ ID NOs: 31-34 and 36-44).

The nucleotide sequences of the miRNAs of the known (conserved) ginger miRNA families MIR319, MIR 159, MIR396, MIR 168, MIR 156 and MIR 164 are reported in Xing H et al. Genome-wide investigation of microRNAs and expression profiles during rhizome development in ginger (Zingiber officinale Roscoe). BMC Genomics. 2022; 23(1):49 (see esp. the Supplementary Table S2 therein) and are reproduced herein in Example 8, Table 1.

In a fifth aspect, the invention relates to the use of the EVs according to the third or fourth aspect of the invention in the manufacture of a beverage, a non-beverage food, a dietary supplement, a drug and/or a cosmetic or personal care product.

The term “cosmetic product”, as used herein, means any substance or preparation suited to be brought into contact with the various surface parts of the human body (epidermis, hair, including body hair, nails, lips and external genital organs) or with the teeth and the oral mucous membranes, for the purpose, exclusively or principally, of cleaning them, of scenting them, of modifying the appearance thereof and/or of correcting body odors and/or of protecting them or of keeping them in good condition.

The term “personal care product”, as used herein, refers to consumer products used in personal hygiene or for beautification. Personal care products include lip balm, cleansing pads, colognes, cotton swabs, cotton pads, deodorant, eye liner, facial tissue, hair clippers, lip gloss, lipstick, lotion, makeup, mouthwash, pomade, perfumes, shampoo, conditioner, talcum powder, shaving cream, skin cream, toilet paper, wet wipes, toothbrushes, toothpaste and the like.

A cosmetic product and a personal care product are to be held distinct from a pharmaceutical product or a pharmaceutical composition. Only the latter is to exert a disease curative or disease preventive effect upon administration to a subject.

It will be appreciated that the herein disclosed EVs and applications thereof are useful in the fields of human medicine and veterinary medicine. Thus, the terms “subject” or “patient”, as interchangeably used herein, refer to any vertebrate including, without limitation, humans and other primates (e.g., chimpanzees and other apes and monkey species), farm animals (e.g., cattle, sheep, pigs, goats and horses), domestic mammals (e.g., dogs and cats), laboratory animals (e.g., rodents such as mice, rats, rabbits, guinea pigs and hamsters), and birds (e.g., domestic, wild and game birds such as chickens, turkeys and other gallinaceous birds, ducks, geese, and the like). In preferred embodiments, the subject is a mammal. In even more preferred embodiments, the subject is a human.

In a sixth aspect, the invention relates to the EVs according to the third or fourth aspect of the invention for use as a medicament.

The EVs of the invention may be formulated for being administered as part of a pharmaceutical composition, e.g., in combination with a pharmaceutically acceptable carrier. The term “pharmaceutically acceptable”, as used herein, refers to those compounds, materials, compositions, and/or dosage forms which are suitable for use in contact with the tissues of human and animal subjects without excessive toxicity, irritation, allergic response, or other problem or complication, commensurate with a reasonable benefit/risk ratio. The term “pharmaceutically acceptable carrier”, as used herein, refers to pharmaceutically acceptable material, composition or vehicle, such as a liquid or solid filler, diluent, excipient, or solvent encapsulating material. Such preparations may routinely contain pharmaceutically acceptable concentrations of salt, buffering agents, preservatives, compatible carriers, and may optionally comprise other (i.e., secondary) therapeutic agents. A pharmaceutically acceptable carrier is a pharmaceutically acceptable material, composition or vehicle, such as a liquid or solid filler, diluent, excipient, solvent or encapsulating material, involved in carrying or transporting a prophylactically or therapeutically active agent. Each carrier must be “acceptable” in the sense of being compatible with the other ingredients of the formulation and not injurious to the subject. Some examples of materials which can serve as pharmaceutically acceptable carriers include, but are not limited to, sugars, such as lactose, glucose and sucrose; glycols, such as propylene glycol; polyols, such as glycerin, sorbitol, mannitol and polyethylene glycol; esters, such as ethyl oleate and ethyl laurate; buffering agents, such as magnesium hydroxide and aluminum hydroxide; pyrogen-free water; isotonic saline; Ringer's solution; ethyl alcohol; phosphate buffer solutions; and other nontoxic compatible substances employed in pharmaceutical formulations.

The EVs, or pharmaceutical composition comprising the EVs, can be prepared in any formulation according to a conventional method. The composition may be formulated, for example, as an oral dosage form (e.g., powder, tablet, capsule, syrup, pill, and granule), or parenteral formulations (e.g., an injection formulation). The composition may also be formulated as a systemic formulation or as a topical formulation.

The EVs, or pharmaceutical composition comprising the EVs, is administered in effective amounts. An effective amount is that amount of an agent that alone stimulates the desired outcome. The absolute amount will depend upon a variety of factors, including the material selected for administration, whether the administration is in single or multiple doses, and individual patient parameters including age, physical condition, size, weight, and the stage of the disease. These factors are well known to those of ordinary skill in the art and can be addressed with no more than routine experimentation.

The EVs useful in the therapeutic and/or preventive methods of the present disclosure can be administered via any mode, such as, but not limited to, localized injection, including catheter administration, systemic injection, intravenous injection, intrauterine injection or parenteral administration. Thus, the EVs according to the invention can be administered systemically (e.g., orally, rectally, parenterally (e.g., intravenously), intramuscularly, intraperitoneally, transdermally (e.g., by a patch), topically (as by powders, ointments, drops or transdermal patch), buccally, or as an oral or nasal spray, by inhalation, subcutaneously or the like), by administration into the central nervous system (e.g., into the brain (e.g., intracerebrally, intraventricularly or intracerebroventricular) or spinal cord or into the cerebrospinal fluid), or any combination thereof.

In particularly preferred embodiments, the EVs of the invention or the pharmaceutical composition comprising the EVs of the invention are/is administered orally, transdermally, transmucosally, transnasally, sublinguinally, subdermally, intraocularly and/or via inhalation smokeless delivery, preferably via oral inhalation, however, nasal inhalation or a combination of oral and nasal inhalation can also be used. In alternative preferred embodiments, the EVs or the pharmaceutical composition comprising the EVs are/is administered rectally, intestinally, parenterally, intramuscularly, subcutaneously, intramedullarily, intrathecally, intraventricularly, intravenously, intraperitoneally, and/or transurethrally.

The EVs, or the pharmaceutical composition comprising the EVs, may be formulated in a unit dosage injectable form (e.g., solution, suspension, or emulsion).

The EVs of the present invention, or pharmaceutical composition comprising the EVs, useful for the herein disclosed therapeutic applications may be suitable for single or repeated administration, including two, three, four, five or more administrations. In some embodiments, the EVs, or pharmaceutical composition comprising the EVs, may be administered continuously. Repeated or continuous administration may occur over a period of several hours (e.g., 1-2, 1-3, 1-6, 1-12, 1-18, or 1-24 hours), several days (e.g., 1-2, 1-3, 1-4, 1-5, 1-6 days, or 1-7 days) or several weeks (e.g., 1-2 weeks, 1-3 weeks, or 1-4 weeks) depending on the severity of the condition being treated. If administration is repeated but not continuous, the time in between administrations may be hours (e.g., 4 hours, 6 hours, or 12 hours), days (e.g., 1 day, 2 days, 3 days, 4 days, 5 days, or 6 days), or weeks (e.g., 1 week, 2 weeks, 3 weeks, or 4 weeks). The time between administrations may be the same or it may differ. As an example, if the symptoms of the disease appear to be worsening, the EVs, or pharmaceutical composition comprising the EVs, may be administered more frequently, and then once the symptoms are stabilized or diminishing the EVs, or pharmaceutical composition comprising the EVs, may be administered less frequently.

For the herein disclosed applications, the EVs or the pharmaceutical composition comprising the EVs, may be employed for repeated administration of low dosage forms of EVs, as well as single administrations of high dosage forms of EVs. Low dosage forms may range from, without limitation, 1-10, 1-25, or 1-50, micrograms per kilogram, while high dosage forms may range from, without limitation, 51-1000 micrograms per kilogram. In some embodiments, a high dosage form may range from 51-100, 100-200, 200-300, 300-400, 400-500, 500-600, 600-700, 700-800, 800-900, or 900-1000 micrograms per kilogram. It will generally be understood by the skilled person that, depending on the severity of the disease, the health of the subject, and the mode/route of administration, inter alia, the single or repeated administration of low or high dose EVs are contemplated by the present disclosure. In some embodiments, the number of EVs may be about 10⁶, 10⁷, 10⁸, 10⁹, 10¹⁰, 10¹¹, or 10¹². In some embodiments, the number of EVs to be administered as part of a single dose may be about 10⁶-10⁷, about 10⁷-10⁸, about 10⁸-10⁹, about 10⁹-10¹⁰, about 10¹⁰-10¹¹, or about 10¹¹-10¹² or even more.

In view of the herein disclosed experimentally observed effects with respect to an improvement of the mitochondrial function (BHI) and thus the bioenergetic profile of the target cells at the highest tested concentration ratio of 1000:1 (EVs vs. PBMCs; see Example 7 and FIG. 8), the skilled person will be able, dependent on the targeted condition, the route of administration and the individual subject to be treated, to identify a suitable dose of the EVs, or the pharmaceutical composition comprising the EVs, for achieving the herein envisaged therapeutic effect. Thus, the dose of the EVs may be selected to be pharmaceutically/therapeutically effective. In a preferred embodiment, the EVs may be applied in a dose ratio of at least 1000:1 relative to the amount of target cells or cells comprised in the targeted tissue.

In a seventh aspect, the invention relates to the EVs according to the sixth aspect of the invention for use in the prevention and/or treatment of a pathological condition selected from the group consisting of a cancerous disease, a cardiovascular disease, a neurodegenerative disease, a metabolic disease, an inflammatory disease, an immune disease and an infectious disease.

In preferred embodiments according to the sixth aspect of the invention, the EVs of the invention are for use in the prevention and/or treatment of an inflammatory disease, wherein said inflammatory disease is preferably a form of colitis. The presence of a corresponding therapeutic effect can be assessed, e.g., by a disease model as described in Example 10.

The term “colitis”, as used herein, refers to an acute or chronic inflammation of the colon, in specific embodiments, the membrane lining of the large bowel. Symptoms of colitis may include abdominal pain, diarrhea, rectal bleeding, painful spasms (tenesmus), lack of appetite, colonic ulcers, fever, and/or fatigue. As used herein, the term “colitis” includes, among others and without limitation, any one or more of the following diseases and disorders: inflammatory bowel disease (IBD), Crohn's disease, ulcerative colitis, collagenous colitis, lymphocytic colitis, ischemic colitis, diversion colitis, Behcet's disease and indeterminate colitis.

In preferred embodiments of the latter embodiments, a preventive/therapeutic effect on said colitis is provided by the capacity of the EVs to promote:
intestinal tissue renewal and/or remodelling; and/or
a modulation of the intestinal stem cell microenvironment; and/or
anti-inflammatory pathways.

Moreover, in view of the herein disclosed unexpected findings (e.g., in Example 7 and FIG. 8) that the EVs obtained by the method of the present invention can advantageously improve the mitochondrial function and bioenergetic status of the target cells (e.g., PBMCs) vis-à-vis the known fact that oxidative stress and mitochondrial dysfunction underlies a broad spectrum of pathological conditions and aging, it can be expected that the EVs of the invention will provide an effective treatment modality for both, prophylactic and therapeutic applications, including regenerative medicine, in particular, for treating or preventing such conditions which occurrence and/or progression is known to be causally linked with mitochondrial dysfunction. Prominent examples of the latter conditions, and which correspond to preferred and particularly contemplated targets for preventive and/or therapeutic applications of the EVs of the invention, are inflammatory diseases and/or neurodegenerative diseases, such as Alzheimer's disease (AD) or Parkinson's disease, or aging.

In preferred embodiments, the neurodegenerative disease is a form of dementia, preferably Alzheimer's disease (AD). The presence of a corresponding therapeutic effect can be assessed, e.g., by a disease model as described in Example 9.

The term “Alzheimer's disease (AD)”, as used herein, refers to a mental deterioration associated with specific degenerative brain disease that is characterized by senile plaques, neuritic tangles and progressive neuronal loss which manifests clinically in progressive memory deficits, confusion, behavioral problems, inability to care for oneself, gradual physical deterioration and, ultimately, death. Means and methods as well as the criteria for diagnosing Alzheimer's disease are known in the art. Patients suffering Alzheimer's disease are typically identified using the NINCDS-ADRDA (National Institute of Neurological and Communicative Disorders and the Alzheimer's Disease and Related Disorders Association) criteria: Clinical Dementia Rating (CDR)=1; Mini Mental State Examination (MMSE) between 16 and 24 points and Medial temporal atrophy (determined by Magnetic Resonance Imaging, MRI)>3 points in Scheltens scale. As used herein, the term “Alzheimer's disease” is intended to include all the stages of the disease, including the stages defined by NINCDS-ADRDA Alzheimer's Criteria; see also Dubois B et al., Lancet Neurol. 2007; 6(8):734-46.

In further preferred embodiments of the EVs according to the third, fourth, sixth or seventh aspect of the invention, the EVs are for use in:
stimulating and/or strengthening the immune defense;
promoting hematopoiesis;
treating inflammation;
promoting health and/or longevity;
enhancing mitochondrial function, preferably for enhancing mitochondrial ATP synthesis;
anti-aging and/or skin regeneration;
reducing oxidative stress, preferably oxidative stress and inflammation in skin pathologies;
reducing oxidative-stress-mediated cell damage, preferably skin damage;
treating and/or preventing neuro-degeneration;
treating and/or preventing a metabolic disorder, preferably selected from diabetes, hypertension, obesity and arteriosclerosis;
treating and/or preventing an acute or chronic respiratory disease (e.g., a lung disorder), preferably selected from asthma and chronic obstructive pulmonary disease (COPD);
treating and/or preventing an eye disorder, preferably selected from astigmatism, dry eyes, and conjunctivitis;
treating and/or preventing a skin disease, preferably selected from atopic eczema and open wounds;
treating and/or preventing an (auto-) immune disorder and/or an allergy;
treating and/or preventing a joint disease;
improving cell function and/or organ function;
promoting (gastro-) intestinal (GI) health and/or amelioration of (gastro-) intestinal (GI) illness;
treating and/or preventing neoplasm;
as an anti-microbial agent;
for promoting hair growth and/or preventing hair loss;
treating and/or preventing an erectile dysfunction and/or for increasing sexual potency;
enhancing physical or mental performance; and/or
a method of diagnosis in vivo and/or prognosis in vivo of a disease, preferably the disease being selected from any of the diseases/disorders referred to herein above.

Also contemplated herein are uses of the EVs according to the third, fourth, sixth or seventh aspect of the invention in a method for in vitro or ex vivo diagnosis and/or prognosis of any of the above-referred medical conditions.

The Figures show

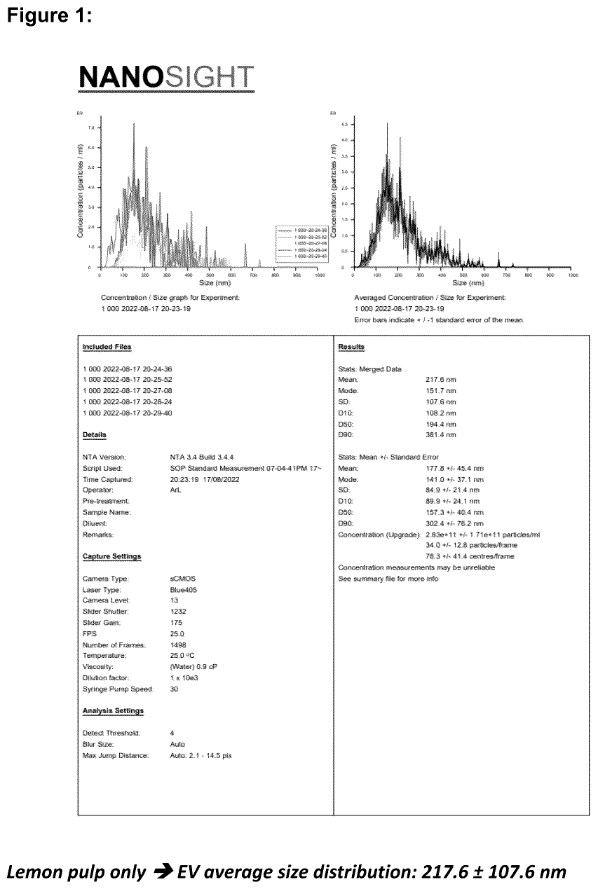

FIG. 1: Nanoparticle tracking analysis (NTA) of extracellular vesicles (EVs) obtained from lemon pulp as described in Example 1 and collected as described in Example 5, showing the number and size distribution. NTA was conducted as described in Example 6.

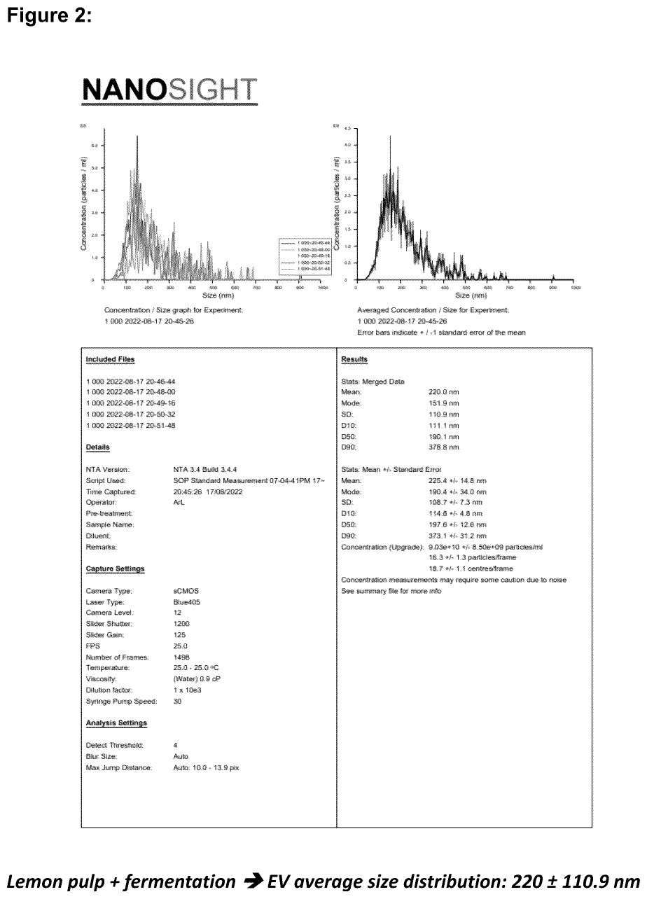

FIG. 2: NTA of EVs obtained from lemon pulp as described in Example 1 which had subsequently been subjected to a fermentation as described in Example 4 and collected as described in Example 5, showing the number and size distribution. NTA was conducted as described in Example 6.

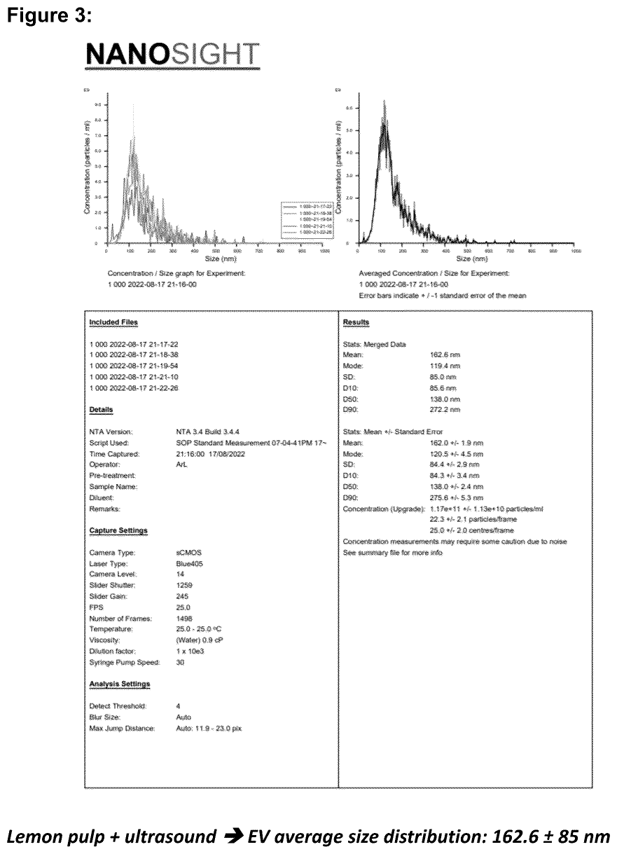

FIG. 3: NTA of EVs obtained from ultrasound-stimulated lemon pulp as described in Example 2 and collected as described in Example 5, showing the number and size distribution. NTA was conducted as described in Example 6.

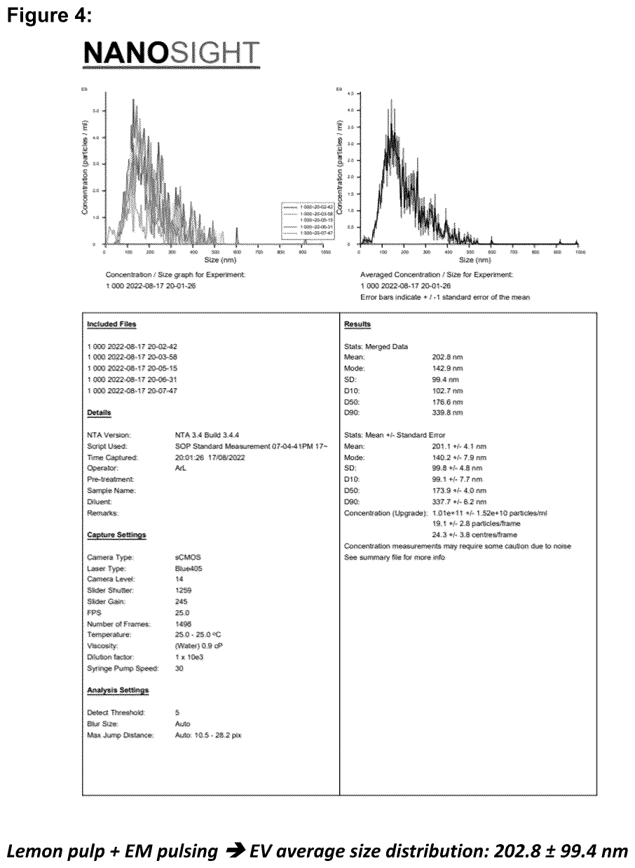

FIG. 4: NTA of EVs obtained from electromagnetic-stimulated lemon pulp as described in Example 3 and collected as described in Example 5, showing the number and size distribution. NTA was conducted as described in Example 6.

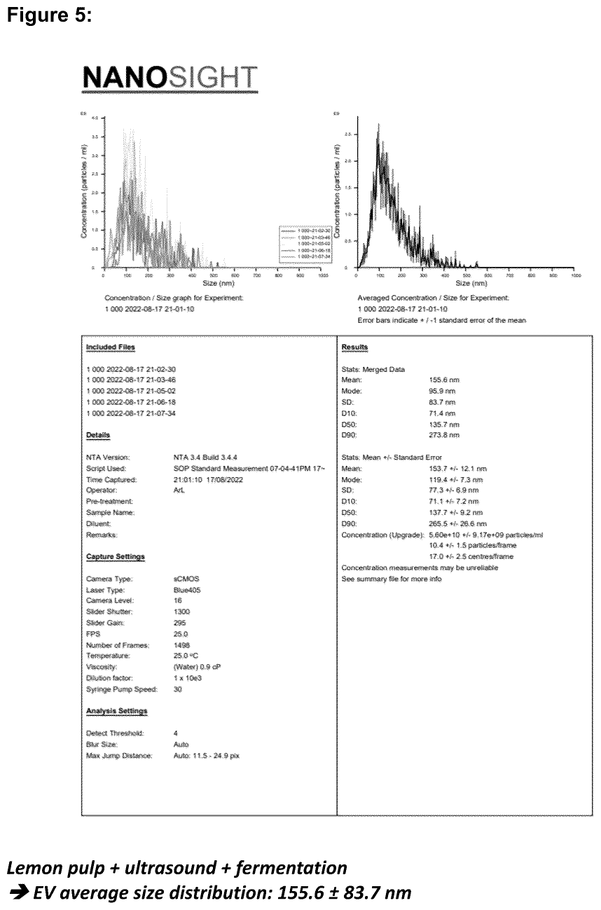

FIG. 5: NTA of EVs obtained from ultrasound-stimulated lemon pulp which had subsequently been subjected to a fermentation as described in Example 4 and collected as described in Example 5, showing the number and size distribution. NTA was conducted as described in Example 6.

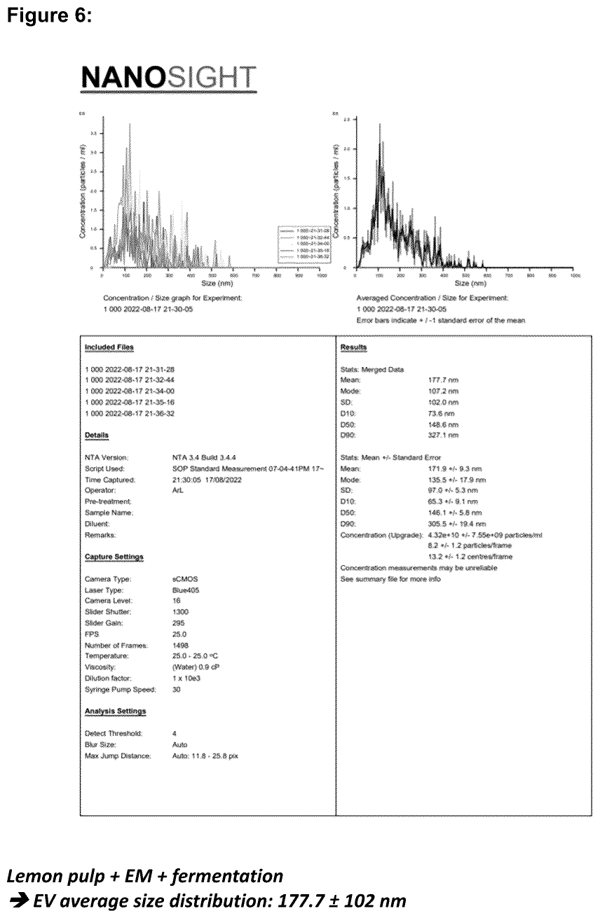

FIG. 6: NTA of EVs obtained from electromagnetic-stimulated lemon pulp which had subsequently been subjected to a fermentation as described in Example 4 and collected as described in Example 5, showing the number and size distribution. NTA was conducted as described in Example 6.

FIG. 7: Bar chart showing a summary of the NTA data of the EVs obtained from the different preparation routes as described in Examples 1 to 5.

FIG. 8: Bar chart of the Bioenergetic Health Index (BHI) data (Example 7) as determined from acquired mitochondrial parameters of peripheral blood mononuclear cells (PBMCs) treated with the EVs from lemon pulp as obtained by the different preparation routes as described in Examples 1 to 3. It is apparent from an increase in the determined BHI values that EVs obtained from acoustically or electromagnetically stimulated lemon pulp, at least when applied in the highest concentration ratio (1000:1) relative to the number of PBMCs, led to a substantial improvement of the mitochondrial function in PBMCs as compared to untreated PBMCs (control) or PBMCs treated by conventionally produced EVs (i.e., EVs isolated from lemon pulp which has not been subjected to an acoustic and/or electromagnetic stimulation).

FIG. 9: Bar chart showing the number of sequence reads (total reads vs. reads corresponding to known and putative miRNAs (18-25 nts length)) from next-generation sequencing (NGS) as obtained from EVs of untreated and differently treated ginger rhizome samples.

The examples illustrate the invention:

### Example 1: Lemon Pulp Preparation

Fresh lemon fruits (Citrus limon) were purchased from a local market peeled, cut in half, and the pulp was homogenized. Prior to peeling, fruits were washed separately under water (about 60-70° C.) and dried with a paper towel in order to remove impurities that could affect the assay result.

### Example 2: In Vitro Ultrasound Stimulation for EV Production from Lemon Pulp

Ultrasound stimulation of the obtained lemon pulp preparation (as described in Example 1) was generated by a transducer at 1-MHz working frequency with a 20% duty cycle, using the Agilent Generator 33220A (Agilent Technologies, Inc., Santa Clara, CA, US). The spatial-peak temporal-average intensity (ISPTA) was 280 mW/cm². Lemon pulp was kept on ice during the ultrasound stimulation and subjected to multiple ultrasound stimulations with 3 min duration per spot. The total sonication time of ultrasound stimulation was 15 min. Subsequently, lemon pulp was stored at 6° C. for 24 hours. Then, the pulp was either fermented for 5 days (as described in Example 4) or immediately centrifuged to collect EVs (as described in Example 5) for being analyzed by nanoparticle tracking analysis (NTA) via NanoSight (as described in Example 6).

### Example 3: In Vitro Electromagnetic (EM) Pulse Stimulation for EV Production from Lemon Pulp

Electromagnetic stimulation of the homogenized lemon pulp prepared as described in Example 1 was conducted by using a Biostim SPT pulse generator (Igea, Carpi, Italy), a generator of pulsed electromagnetic fields (PEMFs) which produced pulses with a damped oscillation of 50 μs (pulse duration) and a base frequency of around 240 kHz. By transferring the energy via plasma chamber, high-frequency oscillation peaks (spikes) of up to 800 MHz arise. An energy output per pulse of around 60 Ws (joule) with a magnetic induction of 100 mT was achieved. Lemon pulp was kept on ice during EM pulse stimulation. The overall time of EM stimulation was 15 minutes with a repetition rate (pulse rate) of 2 Hz. Subsequently, lemon pulp was stored at 6° C. for 24 hours. Then, the pulp was either fermented for 5 days (as described in Example 4) or immediately centrifuged to collect EVs (as described in Example 5) for being analyzed by nanoparticle tracking analysis (NTA) via NanoSight (as described in Example 6).

### Example 4: Fermentation of Lemon Pulp

For fermentation process (anaerobic cultivation), the lemon pulp preparation (as described in Example 1), or the lemon pulp preparations further treated by US or EM (as described in Examples 2 and 3, respectively), were put into a vacuum container together with a 3% (w/v) brine and the fermentation was proceeded at 18-21° for 5 days. Preparing the brine: 30 g sodium chloride per liter water (or 1 oz sodium chloride per qt) for a 3% (w/v) solution.

### Example 5: EV Collection

For collection of the EVs and removal of cells and cellular debris, the lemon pulp preparation (as described in Example 1), or the lemon pulp preparation which was further treated as described in Examples 2 to 4, was subjected to a differential centrifugation, in particular, three sequential centrifugation steps conducted at 300×g for 10 min, 2,000×g for 30 min, and 10,000×g for 30 min, respectively, at 4° C. EVs were precipitated from the final supernatant by addition of ExoQuick-TC (System Biosciences, USA) at a 1:5 ratio (v/v), followed by an incubation overnight at 4° C. and subsequent centrifugation at 3000×g for 60 min. The pellet containing the precipitated EVs was resuspended in PBS for being analyzed nanoparticle tracking analysis (NTA) via NanoSight as described in Example 6.

### Example 6: Nanoparticle Tracking Analysis (NTA) Via NanoSight

Quantification and size determination of the EVs was conducted by nanoparticle tracking analysis (NTA) using the NanoSight NS500 instrument (NanoSight Ltd., Malvern, UK) operated at room temperature and the NTA 3.4 Build 3.4.4 Software (Malvern Panalytical, Malvern, UK).

### Example 7: Improvement of Mitochondrial Function and Cellular Bioenergetics in Human Peripheral Blood Mononuclear Cells (PBMCs) Upon Treatment with the EVs of the Invention

Human peripheral blood mononuclear cells (PBMCs) were isolated from blood samples obtained from healthy test subjects by following standard procedures (see, e.g., Methods section in König et al., 2022).

In order to assess any effect(s) of the extracellular vesicles (EVs) of the invention on the cellular/mitochondrial bioenergetics, EVs from lemon pulp obtained by the different preparation routes as described in Examples 1 to 3, concentrated at about 10¹⁰ per ml, were given to the PMBC samples to result in five different concentration ratios (i.e., ratio of EVs to PBMCs: 1:1000, 1:100, 1:10, 1:1, 0.1:1).

For each sample, the bioenergetic health index (BHI) (see Chacko et al., 2014; Chacko et al., 2015; Koklesova et al., 2022) was determined from recorded mitochondrial/cellular parameters as described by König B et al., 2022 as compared to a control of corresponding PBMC cells that have not been contacted with the EVs.

In brief, cellular bioenergetics of the isolated PBMCs was determined using the extracellular flux analyzer XFe96 (Seahorse Bioscience, Agilent Technologies) and the Seahorse XF Cell Mito Stress Test Kit (Agilent Technologies Germany Gmbh & Co. KG, Waldbronn, Germany). In this assay, the response of cellular oxygen consumption to the sequential addition of mitochondrial inhibitors is used. The final well concentrations (2.5×10⁵ PBMCs) of oligomycin, FCCP and rotenone/antimycin were 3/3/5 μM. Oligomycin inhibits ATP synthase (complex V) and is injected first in the assay following basal measurements. It impacts or decreases electron flow through the electron transport chain (ETC), resulting a reduction in mitochondrial respiration or oxygen consumption rate (OCR). This decrease in OCR is linked to cellular ATP production. Carbonyl cyanide-4 (trifluoromethoxy)phenylhydrazone (FCCP) is an uncoupling agent that collapses the proton gradient and disrupts the mitochondrial membrane potential. It is the 2nd injection following Oligomycin. As a result, electron flow through the ETC is uninhibited, and oxygen consumption by complex IV reaches the maximum. The FCCP-stimulated OCR can then be used to calculate spare respiratory capacity, defined as the difference between maximal respiration and basal respiration. Spare respiratory capacity is a measure of the ability of the cell to respond to increased energy demand or under stress. The third injection is a mixture of rotenone, a complex I inhibitor, and antimycin A, a complex III inhibitor. This combination shuts down mitochondrial respiration and enables the calculation of nonmitochondrial respiration driven by processes outside the mitochondria. The experiments were performed in accordance with the manufacturer instructions (Agilent Technologies) and were replicated in six wells and averaged for each experimental condition. The recorded parameters were 1) Basal oxygen consumption rate (OCR in pmol/min); 2) ATP-linked OCR and proton leak; 3) maximal OCR and reserve capacity; 4). Non-mitochondrial OCR. Finally, the parameters from the mitochondrial stress test (MST) were integrated as a bioenergetic health index (BHI) as described in König et al., 2022 and therein referenced publications by Kramer et al., 2014 and Chacko et al., 2014.

The BHI results determined from the PBMC samples treated with the differentially prepared EVs are illustrated in the bar chart shown in FIG. 8.

As it is evident from these data, treatment of the PBMCs with the EVs obtained from acoustically or electromagnetically stimulated lemon pulp led-at least when applied in the highest concentration ratio tested (1000:1)-to a significant increase of the BHI as compared to untreated PBMCs (#control) or PBMCs treated by equal amounts of conventional EVs as obtained from lemon pulp without an acoustic and/or electromagnetic stimulation.

These results demonstrate that the EVs obtainable by the method of the present invention have the capacity to counteract detrimental effects of oxidative stress and to thereby improve mitochondrial functions, and thus the cellular bioenergetic profile. These findings suggest that the EVs obtained by the method of the invention will provide an effective therapeutic means, either alone or as an integrative and complementary support, for pharmacological therapy in a variety of acute and chronic diseases, in particular, where mitochondrial dysfunction plays a central role, as well as in the regenerative and/or preventive medicine, including anti-aging and promoting longevity.

### Example 8: EVs from Ginger and Analysis of the miRNA Content (Quality and Quantity)

### Sample Preparation

The samples (n=4) used were generated as follows:

### (i) “Untreated” Ginger Rhizomes

### Ginger Extract Preparation

Rhizomes of ginger (Zingiber officinale) were purchased from a local market. To prepare laboratory samples, the rhizomes were homogenized (700 W mixer, 30 sec). These steps were performed immediately after the ginger rhizomes had been purchased. Prior to that, the ginger rhizomes were washed separately under water (about 60-70° C.) and dried with a paper towel in order to remove impurities that could affect the assay result.

### (ii) Fermented Ginger Rhizome

The fermentation of the ginger extract preparation was conducted analogously as described for lemon pulp in Example 4.
   (iii) Sonicated Ginger Rhizome
In vitro ultrasound stimulation of the ginger extract preparation was conducted analogously as described for lemon pulp in Example 2.

### (iv) Sonicated & Fermented Ginger Rhizome

In vitro ultrasound stimulation of the ginger extract preparation followed by fermentation was conducted analogously as described for lemon pulp in Example 2.
   PEG Pulldown for EV Isolation and Subsequent RNA Isolation from EVs:

Then, PEG pulldown of ginger rhizomes-derived EVs was performed according to the protocol described by Kalarikkal et al. Sci Rep. 2020; 10:4456. After PEG pulldown, small RNAs were isolated from the EVs using the exoRNeasy Serum/Plasma Maxi Kit (Qiagen).

The short RNA was then used as a basis for a short RNA sequencing approach with reverse transcription and adaptor ligation followed by size exclusion and ultimately by sequencing on a next generation sequencer (NGS) according to the sequencing-by-synthesis (SBS)-method. Generated raw data was exported to FASTQ data and processed according to a standard pipeline of small RNA sequencing.

The reads were annotated to the ginger genome, the ginger transcriptome and known miRNAs from ginger (Xing H et al. Genome-wide investigation of microRNAs and expression profiles during rhizome development in ginger (Zingiber officinale Roscoe). BMC Genomics. 2022; 23(1):49). Moreover, the found known ginger miRNAs were checked for their seed sequences and compared to the human miRNA database using the online tool TargetScanHuman (v8.0) (https://www.targetscan.org/vert_80/; Agarwal V et al. Predicting effective microRNA target sites in mammalian mRNAs. Elife. 2015; 4:e05005; McGeary S E et al. Science. 2019; 366(6472):eaav1741; Lewis B P et al., Cell. 2005; 120(1):15-20). Additional databases for target genes were consulted resulting in a list that was correlated with biological processes according to pathway and ontology databases.

The sequencing results show that miRNAs (known and putative miRNAs) were comprised in all samples. The EVs from untreated ginger yielded less miRNAs as compared to each of the treated versions, whereas the combination of sonification and fermentation resulted in the highest detected quantities (see FIG. 9). These results demonstrate that each of the treatments (i.e., sonication or fermentation) applied individually advantageously results in an increase of the miRNA content, and that the combined treatment (i.e., sonication and fermentation) synergistically provides a further enhancement with respect to the miRNA quantity.

An analysis of the obtained sequencing results identified for each of the test samples the presence of several conserved ginger miRNAs, among which miRNAs of the six known miRNA families MIR156, MIR159, MIR164, MIR168, MIR319 and MIR396 (cf. Table 1) were detected at highest frequencies. It is noteworthy that currently most ginger miRNAs are not yet annotated or described (see, e.g., Xing H et al. Genome-wide investigation of microRNAs and expression profiles during rhizome development in ginger (Zingiber officinale Roscoe). BMC Genomics. 2022; 23(1):49).

TABLE 1
Conserved miRNAs in ginger (adapted from Supplementary Table S2 of Xing H et al.
Genome-wide investigation of microRNAs and expression profiles during rhizome
development in ginger (Zingiber officinale Roscoe). BMC Genomics. 2022; 23(1): 49).

| miRNA | miRNA family | | |
|---|---|---|---|
| family | member | nucleotide sequence (5′-3′) | |
| MIR156 | miR156 | CUGACAGAAGAGAGUGAGCAC | SEQ ID NO: 1 |
| | miR156_2 | UUGACAGAAGAUAGAGAGC | SEQ ID NO: 2 |
| | miR156a | UGACAGAAGAGAGUGAGCACA | SEQ ID NO: 3 |
| | miR156a_2 | UGACAGAAGAGAGAGAGCAC | SEQ ID NO: 4 |
| | miR156a-5p | UGACAGAAGAGAGUGAGCAC | SEQ ID NO: 5 |
| | miR156c | UUGACAGAAGAGAGAGAGCAC | SEQ ID NO: 6 |
| | miR156c-3p_2 | GCUCACUUCUCUCUCUGUCAGC | SEQ ID NO: 7 |
| | miR 156d-3p_4 | GCUCUCUAUGCUUCUGUCAUCA | SEQ ID NO: 8 |
| | miR156e-3p | GCUCACUGCUCUCUCUGUCAUC | SEQ ID NO: 9 |
| | miR156f-5p | CUGACAGAAGAGAGUGAGCA | SEQ ID NO: |
| | | | 10 |
| | miR156j_1 | GUUGACAGAAGAGAGUGAGCAC | SEQ ID NO: |
| | | | 11 |
| | miR156k_1 | UGACAGAAGAGAGGGAGCAC | SEQ ID NO: |
| | | | 12 |
| | miR 156k_2 | UUGACAGAAGAGAGUGAGCAC | SEQ ID NO: |
| | | | 13 |
| MIR159 | miR159 | UUUGGACUGAAGGGAGCUCUA | SEQ ID NO: |
| | | | 14 |
| | miR159a.1 | UUUGGAUUGAAGGGAGCUCUG | SEQ ID NO: |
| | | | 15 |
| | miR 159a_1 | UUUGGAUUGAAGGGAGCUCUA | SEQ ID NO: |
| | | | 16 |
| | miR159b-5p | AGCUGCCGACUCAUUCAUUCA | SEQ ID NO: |
| | | | 17 |
| MIR164 | miR164a_1 | CACGUGCUCCCCUUCUCCAAC | SEQ ID NO: |
| | | | 18 |
| | miR164a_2 | UGGAGAAGCAGGGCACUUGCU | SEQ ID NO: |
| | | | 19 |
| | miR 164a_4 | UGGAGAAGCAGGGCACGUGCA | SEQ ID NO: |
| | | | 20 |
| | miR 164b | UGGAGAAGCAGGGCACGUGC | SEQ ID NO: |
| | | | 21 |
| | miR 164c_1 | UGGAGAAGCAGGGUACGUGCA | SEQ ID NO: |
| | | | 22 |
| | miR 164c-5p | UGGAGAAGCAGGGCACGUGCG | SEQ ID NO: |
| | | | 23 |
| | miR 164e-5p | UGGAGAAGCAGGGCACGUGCAA | SEQ ID NO: |
| | | | 24 |
| MIR168 | miR 168 | UCGCUUGGUGCAGGUCGGGA | SEQ ID NO: |
| | | | 25 |
| | miR 168-3p | UCCCGCCUUGCAUCAACUGAAU | SEQ ID NO: |
| | | | 26 |
| | miR 168-5p | UCGCUUGGUGCAGGUCGGGAAC | SEQ ID NO: |
| | | | 27 |
| | miR168a-3p | CCCGCCUUGCAUCAACUGAAU | SEQ ID NO: |
| | | | 28 |
| | miR 168a-5p | UCGCUUGGUGCAGGUCGGGAA | SEQ ID NO: |
| | | | 29 |
| | miR 168b_1 | UCGCUUGGUGCAGGUCGGG | SEQ ID NO: |
| | | | 30 |
| MIR319 | miR319_1 | UUGGACUGAAGGGAGCUCC | SEQ ID NO: |
| | | | 31 |
| | miR319a | CUUGGACUGAAGGGAGCUCC | SEQ ID NO: |
| | | | 32 |
| | miR319a_1 | UUGGACUGAAGGGAGCUCCCU | SEQ ID NO: |
| | | | 33 |
| | miR319a-3p | UUGGACUGAAGGGAGCUCCC | SEQ ID NO: |
| | | | 34 |
| | miR319a-5p_1 | AGAGCUUCCUUCAGUCCACUC | SEQ ID NO: |
| | | | 35 |
| | miR319b_1 | CUUGGACUGAAGGGAGCUCCU | SEQ ID NO: |
| | | | 36 |
| | miR319b_2 | UUGGACUGAAGGGUGCUCCC | SEQ ID NO: |
| | | | 37 |
| | miR319b-3p | UUGGACUGAAGGGUGCUCCCU | SEQ ID NO: |
| | | | 38 |
| | miR319c | UUGGACUGAAGGGAGCUCCUU | SEQ ID NO: |
| | | | 39 |
| | miR319c_1 | UUGGACUGAAGGGAGCUCCU | SEQ ID NO: |
| | | | 40 |
| | miR319c_2 | CUUGGACUGAAGGGAGCUCCC | SEQ ID NO: |
| | | | 41 |
| | miR319e_1 | UUUGGACUGAAGGGAGCUCCU | SEQ ID NO: |
| | | | 42 |
| | miR319g_1 | UUGGACUGAAGGGAGCUCCUUC | SEQ ID NO: |
| | | | 43 |
| | miR319q | UGGACUGAAGGGAGCUCCUUC | SEQ ID NO: |
| | | | 44 |
| MIR396 | miR396a-3p_1 | GUUCAAUAAAGCUGUGGGAAG | SEQ ID NO: |
| | | | 45 |
| | miR396a-3p_4 | GUUCAAUAAAGCUGUGGGAA | SEQ ID NO: |
| | | | 46 |
| | miR396a-3p_5 | UUCAAUAAAGCUGUGGGAAG | SEQ ID NO: |
| | | | 47 |
| | miR396a-5p | UUCCACAGCUUUCUUGAACUG | SEQ ID NO: |
| | | | 48 |
| | miR396b | UUCCACAGCUUUCUUGAACU | SEQ ID NO: |
| | | | 49 |
| | miR396b-3p_1 | GUUCAAUAAAGCUGUGGGAAA | SEQ ID NO: |
| | | | 50 |
| | miR396b-3p_3 | GUUCAAGAAAGCCCAUGGAAA | SEQ ID NO: |
| | | | 51 |
| | miR396b-5p | UUCCACAGCUUUCUUGAACUU | SEQ ID NO: |
| | | | 52 |
| | miR396c_3 | UUCCACAGGCUUUCUUGAACUG | SEQ ID NO: |
| | | | 53 |
| | miR396d | CUCCACAGGCUUUCUUGAACUG | SEQ ID NO: |
| | | | 54 |
| | miR396e | UUCCACAGCUUUCUUGAACUGU | SEQ ID NO: |
| | | | 55 |
| | miR396e-5p_1 | UCCACAGGCUUUCUUGAACUG | SEQ ID NO: |
| | | | 56 |
| | miR396f-5p | UCUCCACAGGCUUUCUUGAACU | SEQ ID NO: |
| | | | 57 |
| | miR396g-3p | GUUCAAGAAAGCUGUGGAAGA | SEQ ID NO: |
| | | | 58 |
| | miR396g-5p_1 | UCCCACAGCUUUAUUGAACUG | SEQ ID NO: |
| | | | 59 |
| | miR396h | UCCACAGCUUUCUUGAACUG | SEQ ID NO: |
| | | | 60 |

The top 6 of the identified known (conserved) miRNAs in the order of their frequencies (quantified sequence reads) for each of the samples are given in Table 2.

TABLE 2
Top 6 ranking of detected known (conserved) ginger
miRNAs¹ by frequency of sequence reads (1: highest; 6: lowest)

| | untreated | sonified | fermented | sonified & |
|---|---|---|---|---|
| miRNAs top 6 | ginger | ginger | ginger | fermented ginger |
| 1 | MIR319 | MIR319 | MIR159 | MIR319 |
| 2 | MIR396 | MIR159 | MIR319 | MIR159 |
| 3 | MIR168 | MIR396 | MIR396 | MIR168 |
| 4 | MIR159 | MIR168 | MIR168 | MIR396 |
| 5 | MIR156 | MIR156 | MIR164 | MIR164 |
| 6 | MIR164 | MIR164 | MIR156 | MIR156 |

¹The nucleotide sequences of the known ginger miRNAs are given in Table 1 (reproduced from the Supplementary Table S2 of Xing H et al. BMC Genomics. 2022; 23(1): 49).

In all ginger samples, sequence reads corresponding to miRNAs of the miRNA families MIR156, MIR159, MIR164, MIR168, MIR319 and MIR396 were detected (see above). Dependent on the kind of treatment (fermentation, sonification, or sonification and fermentation), however, their frequency and ranking (relative order of abundance) changed. Therefore, these results demonstrate that each of the different treatments not only results in a quantitative change, but also in a qualitative one. The x-fold changes of the frequencies of the different miRNAs by treatment vs. untreated are depicted in Table 3.

TABLE 3
x-Fold changes in the total number of detected miRNA reads
from given conserved ginger miRNA families upon the different
indicated treatments (vs. untreated sample).

| | due to | due to | sonification & |
|---|---|---|---|
| miRNA | sonification | fermentation | fermentation |
| MIR156 | 11.7 | 9.3 | 24.7 |
| MIR159 | 53.4 | 119.9 | 109.2 |
| MIR164 | 31.3 | 66.7 | 84.5 |
| MIR168 | 10.5 | 8.0 | 21.3 |
| MIR319 | 15.5 | 9.9 | 19.8 |
| MIR396 | 11.8 | 7.4 | 8.9 |

The detected known (conserved) miRNAs from ginger were assessed for sequence similarity to known human miRNAs (hsa-miR). For none of the detected known ginger miRNAs, a complete match of the entirety of the sequence was found. However, for the ginger miRNA family “MIR319” (e.g., miRNA319_1), two putative human analogues (i.e., the human miRNAs hsa-miR-1269a and hsa-miR-1269b) were identified that share the same seed sequence needed for function. The nucleotide sequences of miR319_1, hsa-miR-1269a and hsa-miR-1269b are shown in Table 4.

TABLE 4
The miRNA sequences, as well as the seed sequence
(nts 2-8) of the matched miRNA of ginger
compared to human.

| | nucleotide sequence (seed |
|---|---|
| miRNA | sequence¹ indicated by underline) |
| miR319_1 | UUGGACUGAAGGGAGCUCC |
| | (SEQ ID NO: 31) |
| hsa-miR-1269a | CUGGACUGAGCCGUGCUACUGG |
| | (SEQ ID NO: 61) |
| hsa-miR-1269b | CUGGACUGAGCCAUGCUACUGG |
| | (SEQ ID NO: 62) |

¹The seed sequence is the essential (conserved) part of an miRNA directly binding to the mRNA and thereby actively regulating gene expression.

Potential genetic targets (i.e., the reactome) of the human miRNAs hsa-miR-1269a and hsa-miR-1269b were taken from the miRTarBase (a repository of miRNA targets; see Huang H Y et al., Nucleic Acids Res. 2022; 50(D1):D222-D230) and analyzed for their biological function. This was done by association of Reactome (a pathway ontology repository) via a standard gene enrichment calculation (Haw R. et al. Reactome Pathway Analysis to Enrich Biological Discovery in Proteomics Datasets Proteomics. 2011 September; 11(18):3598-3613). Table 5 lists highly significant pathways associated with the targets of hsa-miR-1269a and hsa-miR-1269b.

TABLE 5
Reactome pathways affected by targets of hsa-miR-1269a
and hsa-miR-1269b in humans with associated p-value

| ID | Description | P value |
|---|---|---|
| R-HSA-195721 | cell fate decision and | 3.51E−14 |
| | stem cell maintenance | |
| R-HSA-73894 | anti-senescence and | 3.53E−13 |
| | anti-inflammation | |
| R-HSA-2559583 | tumor-suppressive, | 2.33E−12 |
| | cell fate decision | |
| R-HSA-168255 | anti-viral immune response | 4.32E−11 |
| R-HSA-9679506 | anti-viral immune response | 1.64E−09 |
| R-HSA-2559580 | tumor-suppressive, | 2.29E−08 |
| | cell fate decision | |
| R-HSA-9694516 | anti-viral immune response | 3.37E−08 |
| R-HSA-162906 | anti-viral immune response | 5.61E−08 |
| R-HSA-109581 | tissue homeostasis, | 1.95E−07 |
| | cell fate decision | |
| R-HSA-5357801 | tumor-suppressive, | 2.31E−07 |
| | tissue homeostasis | |

In consequence of an uptake of ginger miRNAs (or ginger-derived EVs comprising such miRNAs) by human cells and resulting cross-kingdom RNA interference, effects on cellular senescence, tissue homeostasis, anti-inflammation, tumor-suppression, and anti-viral immune response can be expected (see Table 5). This is in high accordance with recent literature on ginger-derived exosomes which therein were shown to provide comparable anti-fibrosis, anti-virus, and anti-tumor effects as mammalian-derived exosomes:
Teng Y. et al. Plant-derived exosomal microRNAs inhibit lung inflammation induced by exosomes SARS-CoV-2 Nsp12. Mol Ther. 2021 Aug. 4; 29(8): 2424-2440.
Mu J. et al. Interspecies communication between plant and mouse gut host cells through edible plant derived exosome-like nanoparticles. Mol Nutr Food Res. 2014 July; 58(7): 1561-1573.
Kumar A. et al. Ginger nanoparticles mediated induction of Foxa2 prevents high-fat diet-induced insulin resistance. Theranostics. 2022; 12(3): 1388-1403.
Zhang M. et al. Edible Ginger-Derived Nanoparticles: A Novel Therapeutic Approach for the Prevention and Treatment of Inflammatory Bowel Disease and Colitis-Associated Cancer. Biomaterials. 2016 September; 101:321-340.
Zhuang X. et al. Ginger-derived nanoparticles protect against alcohol-induced liver damage. J Extracell Vesicles. 2015; 4:28713.
Chen X. et al. Exosome-like Nanoparticles from Ginger Rhizomes Inhibited NLRP3 Inflammasome Activation. Mol Pharm. 2019 Jun. 3; 16(6):2690-2699.
Kim J. et al. Plant-derived exosome-like nanoparticles and their therapeutic activities. Asian J Pharm Sci. 2022 January; 17(1): 53-69.

Notably, the miRNAs of the conserved ginger miRNA family MIR319 were especially found to be changed in frequency upon treatment by sonication, as well as by fermentation. Moreover, the combinatory manipulation (i.e., sonication and fermentation) was found to provide a further improvement in terms of the quantity of known and putative miRNAs comprised in the thereby obtained EVs.

### Example 9: Plant-Derived (Ginger) EVs in an In Vivo Alzheimer's Disease Model

### Background

Dementia is defined clinically as a decline in memory and impaired thinking ability, which are two domains of cognition. Alzheimer's disease (AD) is the most common cause of dementia (Knopman et al., Nat Rev Dis Primers. 2021 May 13; 7(1):33). AD is characterized by memory loss and personality changes, leading to dementia. The pathological hallmarks of AD are senile plaques and neurofibrillary tangles, which comprise abnormally aggregated β-amyloid peptide (Aβ) and hyperphosphorylated tau protein.

### The APP/PS1 Mouse Model of Alzheimer's Disease

This transgenic model features progressive, age-dependent, beta-amyloid (AB) pathology. These APP/PS1 mice are often used as a model for Alzheimer's disease, including cerebral amyloid angiopathy (CAA) (Malm T et al., Int J Alzheimers Dis. 2011; 2011:517160). The APP/PS1 mice display a variety of clinically relevant AD-like symptoms, including increased parenchymal AB load, inflammation, deficits in the cholinergic system, and cognitive impairment.

### Method

Six-month-old male APP/PS1 mice will be divided into three groups, 10 mice/group: A) control group (“H₂O group”); B) group to be treated with plant-derived (e.g., ginger) EVs (“ginger group”); and C) group to be treated with ultrasound-modified plant-derived (e.g., ginger) EVs (“US+ginger group”). The H₂O group (A) will not be treated and drink only untreated water for the period of the experimentation (four weeks). The mice of the other groups (B+C) will be treated with plant-derived EVs (+/−US) for four weeks via gavage administration. The daily dose/mouse will be 6×10⁹ EVs, corresponding to 2×10⁸ PDE/g mouse weight, dissolved in 200 μL of H₂O, administered via gavage.

### Read Out

After 4 weeks, we will investigate the cognitive deficits and the deposition of amyloid-β (Aβ) in the brains of three groups of mice. In addition, phosphorylation (“activation”) of tau protein and the levels of Aβ40 and Aβ42 will be measured. Also, synaptic plasticity will be measured by determination of postsynaptic density protein 95 (PSD-95) and synapsin I and NLRP3 expression. Inflammation markers such as COX-2 and CD11b will also be measured.

### Mode of Action

The structure-activity relationships of ginger phytochemicals show that ginger can be a candidate to treat Alzheimer's Disease (AD) by targeting different ligand sites, e.g., the nucleotide-binding domain and leucine-rich repeat-containing family, pyrin domain-containing 3 (NLRP3) inflammasome. NLRP3 is a key regulator of innate immune responses, and its aberrant activation is highly implicated in the pathogenesis of many diseases such as Alzheimer's disease and type 2 diabetes (Liang et al., 2022; see below).

It was already shown that EVs from ginger rhizomes strongly inhibited, e.g., NLRP3 inflammasome activation (Chen X et al., 2019; see below). The treatment using ginger EVs suppressed pathways downstream of inflammasome activation including caspase 1 autocleavage, interleukin (IL)-1B and IL-18 secretion, and pyroptotic cell death. Consequently, apoptotic speck protein containing a caspase recruitment domain (ASC) oligomerization and speck formation assays indicated that ginger EVs blocked assembly of the NLRP3 inflammasome, thereby influencing the pathogenesis of Alzheimer's and other types of dementia.

Moreover, ginger-derived EVs are known to be neuroprotective in Alzheimer's by containing the bioactive constituents of ginger, 6-gingerol and 6-shogaol, in addition to normal EV components. 6-shogaol leads to CysLT1R-mediated inhibition of cathepsin B. In addition, ginger-derived EVs are able to decrease pro-inflammatory cytokines such as TNF-α, IL-6, and IL-1b and increased anti-inflammatory cytokines IL-10 and IL-22 with additional neuroprotective effects (Aghajanpour et al., 2017; see below).

Consequently, we expect a higher therapeutic effect in the Alzheimer's in vivo model by the ultrasound-stimulated EVs as compared to unstimulated EVs from ginger, leading to, e.g., higher anti-inflammatory effects, as well as higher inhibitory effects of NLRP3- and/or CysLT1R-pathway.

### LITERATURE

Liang, T., Zhang, Y., Wu, S., Chen, Q., and Wang, L. (2022). The role of NLRP3 inflammasome in Alzheimer's disease and potential therapeutic targets. Front. Pharmacol. 13:845185. doi: 10.3389/fphar.2022.845185
Xingyi Chen, You Zhou, Jiujiu Yu. Exosome-like Nanoparticles from Ginger Rhizomes Inhibited NLRP3 Inflammasome Activation. Mol Pharm. 2019 Jun. 3; 16(6):2690-2699.
Mohammad Aghajanpour, Mohamad Reza Nazer, Zia Obeidavi, Mohsen Akbari, Parya Ezati, and Nasroallah Moradi Kor. Functional foods and their role in cancer prevention and health promotion: a comprehensive review. Am J Cancer Res. 2017; 7(4): 740-769.
Ji-Young Na, Kibbeum Song, Ju-Woon Lee, Sokho Kim, Jungkee Kwon. 6-Shogaol has anti-amyloidogenic activity and ameliorates Alzheimer's disease via CysLT1R-mediated inhibition of cathepsin B. Biochem Biophys Res Commun. 2016 Aug. 12; 477(1):96-102.

### Example 10: Plant-Derived (Grape) EVs in an In Vivo Colitis Disease Model

Colitis will be induced in C57BL/6 mice either by oral infection with Citrobacter rodentium or by DSS (dextran sodium sulphate) administration (see Bettenworth et Thoennissen et al., Molecular Nutrition & Food Research, 2014; 58(7):1474-90).

Method for C. rodentium- or DSS-Induced Colitis

Six-month-old male C57BL/6 mice will be orally challenged with
i) 10⁸-109 cfu of C. rodentium or
ii) DSS 1% for a period of 7 days,
   and divided into three groups, 10 mice/group: A) control group (“H₂O group”); B) group to be treated with plant-derived (e.g., grapes) EVs (“grape group”); and C) group to be treated with ultrasound-modified plant-derived (e.g., grapes) EVs (“US+grape group”). The H₂O group (A) will not be treated and drink only untreated water for the period of the experimentation (7-12 days). The mice of the other groups (B+C) will be treated with plant-derived EVs (+/−US) for 7-12 days via gavage administration. The daily dose/mouse will be 6×10⁹ EVs, corresponding to 2×10⁸ PDE/g mouse weight, dissolved in 200 μL of H₂O, administered via gavage.

### Read Out

In the C. rodentium-induced colitis experiment, we will measure systemic bacterial invasion, histological colon damage and fecal clearance of C. rodentium. Moreover, in the DSS colitis model, we will assess body weight, histological damage, and myeloperoxidase activity.

### Mode of Action

It has already been shown that EVs derived from grapes not only modulate intestinal tissue renewal processes in colitis, but can participate in the remodeling of it in response to pathological triggers by modulating stem cell microenvironment and anti-inflammatory pathways (Ayyar et al., 2021; Ghiasi et al., 2018; Ju et al., 2013; see below).

Consequently, we expect significantly more intestinal tissue renewal, less histological damage, and higher fecal clearance of C. rodentium in the mice treated with the ultrasound-stimulated versus unstimulated EVs from grapes. Explanation for the expected read out could be higher amount of anti-inflammatory, and intestinal stem cell activating miRNA.

### LITERATURE

Rahimi Ghiasi, Moosa; Rahimi, Elnaz; Amirkhani, Zohreh; Salehi, Rasoul. Leucine-rich Repeat-containing G-protein Coupled Receptor 5 Gene Overexpression of the Rat Small Intestinal Progenitor Cells in Response to Orally Administered Grape Exosome-like Nanovesicles. Advanced Biomedical Research 2018; 7(1):p 125.
Songwen Ju et al. Grape Exosome-like Nanoparticles Induce Intestinal Stem Cells and Protect Mice From DSS-Induced Colitis. Molecular Therapy, Volume 21, Issue 7, July 2013, Pages 1345-1357.
Kanchana K. Ayyar, Alan C. Moss Front. Exosomes in Intestinal Inflammation. Pharmacol., 9 Jun. 2021, Vol. 12-2021.

### Example 11: EVs from Spirulina Platensis

Product: approx. 250 g of Spirulina Platensis per liter of water (purchased from officially licenzed producer).

### Treatment:

In vitro ultrasound stimulation of the Spirulina Platensis cells was conducted analogously (using same parameters) as described for lemon pulp in Example 2.

### Collection of EVs:

Collection of EVs was conducted analogously as described for lemon pulp in Example 5.

### Read Out:

It is expected by the inventors that the present technology when applied in connection with Spirulina Platensis cells will also, similarly as observed for lemon pulp and/or ginger, provide an enhancement in the production of EVs, in terms of both, their quantity and their quality (reduced mean size and altered content, esp. an increased miRNA content). It is also expected that corresponding EVs have a similar capacity to provide an improvement of the bioenergetic function of mitochondria (as demonstrated for EVs from lemon pulp in Example 7).

### FURTHER REFERENCES

Yu, D., Li, Y., Wang, M. et al. Exosomes as a new frontier of cancer liquid biopsy. Mol Cancer 21, 56 (2022).
Bhatti, Jasvinder Singh, et al. “Exosome nanocarriers: A natural, novel, and perspective approach in drug delivery system.” Nanoarchitectonics in Biomedicine. William Andrew Publishing, 2019. 189-218.
Zhao Y, Liu T, Zhou M. Immune-Cell-Derived Exosomes for Cancer Therapy. Mol Pharm. 2022 Sep. 5; 19(9):3042-3056. doi: 10.1021/acs.molpharmaceut.2c00407. Epub 2022 Jul. 25. PMID: 35876318.
Zheng Y, Hasan A, Nejadi Babadaei M M, Behzadi E, Nouri M, Sharifi M, Falahati M. Exosomes: Multiple-targeted multifunctional biological nanoparticles in the diagnosis, drug delivery, and imaging of cancer cells. Biomed Pharmacother. 2020 September; 129:110442. doi: 10.1016/j.biopha.2020.110442. Epub 2020 Jun. 24. PMID: 32593129.
Lorenc T, Chrzanowski J, Olejarz W. Current Perspectives on Clinical Use of Exosomes as a Personalized Contrast Media and Theranostics. Cancers (Basel). 2020 Nov. 16; 12(11):3386. doi: 10.3390/cancers12113386. PMID: 33207614; PMCID: PMC7698051.
Urzi O, Raimondo S, Alessandro R. Extracellular Vesicles from Plants: Current Knowledge and Open Questions. Int J Mol Sci. 2021 May 20; 22(10):5366. doi: 10.3390/ijms22105366. PMID: 34065193; PMCID: PMC8160738.
Urzi O, Gasparro R, Ganji N R, Alessandro R, Raimondo S. Plant-RNA in Extracellular Vesicles: The Secret of Cross-Kingdom Communication. Membranes (Basel). 2022 March 23; 12(4):352. doi: 10.3390/membranes12040352. PMID: 35448322; PMCID: PMC9028404.
McClements, D. Julian. “Advances in the application of ultrasound in food analysis and processing.” Trends in Food Science & Technology 6.9 (1995): 293-299.
Karamanidou T, Tsouknidas A. Plant-Derived Extracellular Vesicles as Therapeutic Nanocarriers. Int J Mol Sci. 2021 Dec. 24; 23(1):191. doi: 10.3390/ijms23010191. PMID: 35008617; PMCID: PMC8745116.
Cai Y, Zhang L, Zhang Y, Lu R. Plant-Derived Exosomes as a Drug-Delivery Approach for the Treatment of Inflammatory Bowel Disease and Colitis-Associated Cancer. Pharmaceutics. 2022 Apr. 8; 14(4):822. doi: 10.3390/pharmaceutics14040822. PMID: 35456656; PMCID: PMC9029273.
Bhargava, Nitya, et al. “Advances in application of ultrasound in food processing: A review.” Ultrasonics sonochemistry 70 (2021): 105293.
Mitragotri S. Healing sound: the use of ultrasound in drug delivery and other therapeutic applications. Nat Rev Drug Discov. 2005 March; 4(3):255-60. doi: 10.1038/nrd1662. PMID: 15738980.
Amaroli A, Chessa M G, Bavestrello G, Bianco B. Effects of an extremely low-frequency electromagnetic field on stress factors: a study in Dictyostelium discoideum cells. Eur J Protistol. 2013 August; 49(3):400-5
Kim H J, Jung J, Park J H, Kim J H, Ko K N, Kim C W. Extremely low-frequency electromagnetic fields induce neural differentiation in bone marrow derived mesenchymal stem cells. Exp Biol Med (Maywood). 2013 Aug. 1; 238(8):923-31. doi: 10.1177/1535370213497173. PMID: 23970408.
Hamid H A, Sarmadi V H, Prasad V, Ramasamy R, Miskon A. Electromagnetic field exposure as a plausible approach to enhance the proliferation and differentiation of mesenchymal stem cells in clinically relevant scenarios. J Zhejiang Univ Sci B. 2022 Jan. 15; 23(1):42-57. doi: 10.1631/jzus.B2100443. PMID: 35029087; PMCID: PMC8758935.
Wong C J K, Tai Y K, Yap J L Y, Fong C H H, Loo L S W, Kukumberg M, Fröhlich J, Zhang S, Li J Z, Wang J W, Rufaihah A J, Franco-Obregón A. Brief exposure to directionally-specific pulsed electromagnetic fields stimulates extracellular vesicle release and is antagonized by streptomycin: A potential regenerative medicine and food industry paradigm. Biomaterials. 2022 Jul. 6; 287:121658. doi: 10.1016/j.biomaterials.2022.121658. Epub ahead of print. PMID: 35841726.
Ross C L, Siriwardane M, Almeida-Porada G, Porada C D, Brink P, Christ G J, et al. The effect of low-frequency electromagnetic field on human bone marrow stem/progenitor cell differentiation. Stem Cell Res. 2015; 15:96-10⁸.
Yuana Y, Jiang L, Lammertink B H A, Vader P, Deckers R, Bos C, Schiffelers R M, Moonen C T. Microbubbles-Assisted Ultrasound Triggers the Release of Extracellular Vesicles. Int J Mol Sci. 2017 Jul. 25; 18(8):1610. doi: 10.3390/ijms18081610. PMID: 28757579; PMCID: PMC5578002.
Yuana Y, Balachandran B, van der Wurff-Jacobs K M G, Schiffelers R M, Moonen C T. Potential Use of Extracellular Vesicles Generated by Microbubble-Assisted Ultrasound as Drug Nanocarriers for Cancer Treatment. Int J Mol Sci. 2020 Apr. 24; 21(8):3024. doi: 10.3390/ijms21083024. PMID: 32344752; PMCID: PMC7216118.
Deng Z, Wang J, Xiao Y, Li F, Niu L, Liu X, Meng L, Zheng H. Ultrasound-mediated augmented exosome release from astrocytes alleviates amyloid-β-induced neurotoxicity. Theranostics. 2021 Feb. 25; 11(9):4351-4362. doi: 10.7150/thno.52436. PMID: 33754065; PMCID: PMC7977450.
Mansourian M, Shanei A. Evaluation of Pulsed Electromagnetic Field Effects: A Systematic Review and Meta-Analysis on Highlights of Two Decades of Research In Vitro Studies. Biomed Res Int. 2021 Jul. 29; 2021:6647497. doi: 10.1155/2021/6647497. PMID: 34368353; PMCID: PMC8342182.
Caliogna L, Medetti M, Bina V, Brancato A M, Castelli A, Jannelli E, Ivone A, Gastaldi G, Annunziata S, Mosconi M, Pasta G. Pulsed Electromagnetic Fields in Bone Healing: Molecular Pathways and Clinical Applications. Int J Mol Sci. 2021 Jul. 9; 22(14):7403. doi: 10.3390/ijms22147403. PMID: 34299021; PMCID: PMC8303968.
Kubat N J, Moffett J, Fray L M. Effect of pulsed electromagnetic field treatment on programmed resolution of inflammation pathway markers in human cells in culture. J Inflamm Res. 2015 February 23; 8:59-69. doi: 10.2147/JIR.S78631. PMID: 25759595; PMCID: PMC4346366.
Ambattu L A, Ramesan S, Dekiwadia C, Hanssen E, Li H, Yeo L Y. High frequency acoustic cell stimulation promotes exosome generation regulated by a calcium-dependent mechanism. Commun Biol. 2020 Oct. 5; 3(1):553. doi: 10.1038/s42003-020-01277-6. PMID: 33020585; PMCID: PMC7536404

Momen-Heravi F, Balaj L, Alian S, Mantel P Y, Halleck A E, Trachtenberg A J, Soria C E, Oquin S, Bonebreak C M, Saracoglu E, Skog J, Kuo W P. Current methods for the isolation of extracellular vesicles. Biol Chem. 2013 October; 394(10):1253-62. doi: 10.1515/hsz-2013-0141. PMID: 23770532; PMCID: PMC7075462.
Doyle L M, Wang M Z. Overview of Extracellular Vesicles, Their Origin, Composition, Purpose, and Methods for Exosome Isolation and Analysis. Cells. 2019 Jul. 15; 8(7):727. doi: 10.3390/cells8070727. PMID: 31311206; PMCID: PMC6678302.
Sunkara V, Woo H K, Cho Y K. Emerging techniques in the isolation and characterization of extracellular vesicles and their roles in cancer diagnostics and prognostics. Analyst. 2016 Jan. 21; 141(2):371-81. doi: 10.1039/c5an01775k. PMID: 26535415.
Macia L, Nanan R, Hosseini-Beheshti E, Grau G E. Host- and Microbiota-Derived Extracellular Vesicles, Immune Function, and Disease Development. Int J Mol Sci. 2019 Dec. 22; 21(1):107. doi: 10.3390/ijms21010107. PMID: 31877909; PMCID: PMC6982009.
Rodovalho V R, Luz B S R, Rabah H, do Carmo F L R, Folador E L, Nicolas A, Jardin J, Briard-Bion V, Blottière H, Lapaque N, Jan G, Le Loir Y, de Carvalho Azevedo V A and Guédon E (2020) Extracellular Vesicles Produced by the Probiotic Propionibacterium freudenreichii CIRM-BIA 129 Mitigate Inflammation by Modulating the NF-κB Pathway. Front. Microbiol. 11:1544. doi: 10.3389/fmicb.2020.01544.
Swain M R, Anandharaj M, Ray R C, Parveen Rani R. Fermented fruits and vegetables of Asia: a potential source of probiotics. Biotechnol Res Int. 2014; 2014:250424. doi: 10.1155/2014/250424. Epub 2014 May 28. PMID: 25343046; PMCID: PMC4058509.
Di Cagno R, Coda R, De Angelis M, Gobbetti M. Exploitation of vegetables and fruits through lactic acid fermentation. Food Microbiol. 2013 February; 33(1):1-10. doi: 10.1016/j.fm.2012.09.003. Epub 2012 Sep. 17. PMID: 23122495.
Liu F, Vermesh O, Mani V, Ge T J, Madsen S J, Sabour A, Hsu E C, Gowrishankar G, Kanada M, Jokerst J V, Sierra R G, Chang E, Lau K, Sridhar K, Bermudez A, Pitteri S J, Stoyanova T, Sinclair R, Nair V S, Gambhir S S, Demirci U. The Exosome Total Isolation Chip. ACS Nano. 2017 Nov. 28; 11(11):10712-10723. doi: 10.1021/acsnano.7b04878. Epub 2017 Nov. 1. PMID: 29090896; PMCID: PMC5983373.
König B, Lahodny J. Ozone high dose therapy (OHT) improves mitochondrial bioenergetics in peripheral blood mononuclear cells. Transl Med Commun. 2022; 7(1):17. doi: 10.1186/s41231-022-00123-7. Epub 2022 Jul. 21. PMID: 35880042; PMCID: PMC9301618.
Chacko B K, Kramer P A, Ravi S, Benavides G A, Mitchell T, Dranka B P, Ferrick D, Singal A K, Ballinger S W, Bailey S M, Hardy R W, Zhang J, Zhi D, Darley-Usmar V M. The Bioenergetic Health Index: a new concept in mitochondrial translational research. Clin Sci (Lond). 2014 September; 127(6):367-73. doi: 10.1042/CS20140101. PMID: 24895057; PMCID: PMC4202728.
Chacko B K, Zhi D, Darley-Usmar V M, Mitchell T. The Bioenergetic Health Index is a sensitive measure of oxidative stress in human monocytes. Redox Biol. 2016 August; 8:43-50. doi: 10.1016/j.redox.2015.12.008. Epub 2015 Dec. 24. PMID: 26748041; PMCID: PMC4712317.
Koklesova L, Mazurakova A, Samec M, Kudela E, Biringer K, Kubatka P, Golubnitschaja O. Mitochondrial health quality control: measurements and interpretation in the framework of predictive, preventive, and personalized medicine. EPMA J. 2022 May 12; 13(2):177-193. doi: 10.1007/s13167-022-00281-6. PMID: 35578648; PMCID: PMC9096339.
Kramer P A, Ravi S, Chacko B, Johnson M S, Darley-Usmar V M. A review of the mitochondrial and glycolytic metabolism in human platelets and leukocytes: implications for their use as bioenergetic biomarkers. Redox Biol. 2014; 10(2):206-10.
Kalarikkal S P, Prasad D, Kasiappan R, Chaudhari S R and Sundaram G M: A cost-effective polyethylene glycol-based method for the isolation of functional edible nanoparticles from ginger rhizomes. Sci Rep. 2020; 10:4456.
Huang H Y, Lin Y C, Cui S, Huang Y, Tang Y, Xu J, Bao J, Li Y, Wen J, Zuo H, Wang W, Li J, Ni J, Ruan Y, Li L, Chen Y, Xie Y, Zhu Z, Cai X, Chen X, Yao L, Chen Y, Luo Y, LuXu S, Luo
M, Chiu C M, Ma K, Zhu L, Cheng G J, Bai C, Chiang Y C, Wang L, Wei F, Lee T Y, Huang H D. miRTarBase update 2022: an informative resource for experimentally validated miRNA-target interactions. Nucleic Acids Res. 2022 Jan. 7; 50(D1):D222-D230. doi: 10.1093/nar/gkab1079. PMID: 34850920; PMCID: PMC8728135.

It is understood that the definitions and embodiments as described above in connection with the first aspect of the invention also apply, in as far as possible, mutatis mutandis to the second, third, fourth, fifth, sixth and seventh aspects of the present invention.

The invention is herein described, by way of example only, with reference to the accompanying drawings for purposes of illustrative discussion of the preferred embodiments of the present invention.

Unless otherwise defined, all technical and scientific terms used herein have the same meaning as commonly understood by one of ordinary skill in the art to which this invention belongs. In case of conflict, the patent specification, including definitions, will prevail.

Regarding the embodiments characterized in this specification, in particular in the claims, it is intended that each embodiment mentioned in a dependent claim is combined with each embodiment of each claim (independent or dependent) said dependent claim depends on. For example, in case of an independent claim 1 reciting 3 alternatives A, B, and C, a dependent claim 2 reciting 3 alternatives D, E, and F and a claim 3 dependent on claims 1 and 2 and reciting 3 alternatives G, H, and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2, and 1. In case a further dependent claim 4 is present which refers to any one of claims 1 to 3, it follows that the combination of the subject-matter of claims 4 and 1, of claims 4, 2, and 1, of claims 4, 3, and 1, as well as of claims 4, 3, 2, and 1 is clearly and unambiguously disclosed.

The above considerations apply mutatis mutandis to all appended claims.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

Modifications may be made to the foregoing without departing from the basic aspects of the technology. Although the technology has been described in substantial detail with reference to one or more specific embodiments, those of ordinary' skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation and use of such terms and expressions do not exclude any equivalents of the features shown and described or portions thereof, and various modifications are possible within the scope of the technology claimed. The terms “method” and “process” are used interchangeably herein.

The term “a” or “an” can refer to one of or a plurality of the elements it modifies (e.g., “a cell” can mean “one or more cells”) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

1. A method for enhancing the production of extracellular vesicles (EVs) from non-mammalian cells, the method comprising:
(a) subjecting the cells to an acoustic and/or electromagnetic stimulation; and
(b) optionally, collecting the EVs produced from the cells after the stimulation.

2. The method of claim 1, wherein the non-mammalian cells comprise, consist essentially of, or consist of algae cells, plant cells and/or at least one microorganism.

3. The method of claim 2, wherein the non-mammalian cells comprise, consist essentially of, or consist of plant cells and at least one microorganism selected from the genus Lactobacillus.

4. The method of claim 2, wherein the non-mammalian cells are comprised in a maintenance or culture medium, optionally a maintenance or culture medium comprising, consisting essentially of, or consisting of an aqueous solution or water.

5. The method of claim 1, further comprising, after the stimulation in step (a), and before the optional collecting in step (b), an incubation;
wherein optionally the incubation is conducted:
(i) at a temperature in the range of between, optionally, 1° C. and 42° C., 1° C. and 37° C., 1° C. and 35° C., 1° C. and 30° C., 1° and 25°, 1° C. and 20° C., 1° C. and 15° C., 1° C. and 10° C., 2° C. and 9° C., 3° C. and 8° C., 4° C. and 7° C., optionally at about 6° C.; and/or
(ii) for a time period in the range of between, optionally, 1 hour to 72 hours, 2 hours to 60 hours, 6 hours to 48 hours, 12 hours to 36 hours, 18 hours to 30 hours, 20 hours to 28 hours, optionally about 24 hours.

6. The method of claim 1, wherein the collecting in (b) is conducted by:
(i) centrifugation, thereby obtaining a centrifugation supernatant comprising the EVs;
(ii) precipitation of the EVs from the centrifugation supernatant by contacting the supernatant with a water excluding polymer;
(iii) pelleting the precipitated EVs by centrifugation and disposal of the centrifugation supernatant; and
(iv) resuspending the pelleted EVs in an aqueous buffer.

7. The method of claim 1, wherein the acoustic stimulation in (a) is conducted by applying a continuous or pulsed, optionally pulsed, ultrasound:
(i) at a frequency in the range of between, optionally, 20 kHz and 10 MHz, 25 kHz and 9 MHz, 50 kHz and 8 MHz, 100 kHz and 7 MHz, 250 kHz and 6 MHz, 500 kHz and 4 MHz, 1000 kHz and 2 MHz, 0.2 MHz and 1.8 MHz, 0.4 MHz and 1.6 MHz, 0.6 MHz and 1.4 MHz, 0.8 MHz and 1.2 MHz, optionally at a frequency of about 1 MHz;
(ii) with an intensity in the range of between, optionally, 0.5 mW/cm² and 3 W/cm², 10 mW/cm² and 750 mW/cm², 10 mW/cm² and 750 mW/cm², 50 mW/cm² and 600 mW/cm², 100 mW/cm² and 500 mW/cm², 200 mW/cm² and 360 mW/cm², 230 mW/cm² and 330 mW/cm², 260 mW/cm² and 300 mW/cm², optionally of about 280 mW/cm²;
(iii) with a duty cycle in the range of between, optionally, 1% and 100%, 2% and 90%, 3% and 80%, 4% and 70%, 5% and 60%, 6% and 50%, 7% and 40%, 8% and 35%, 10% and 30%, 15% and 25%, optionally about 20%;
(iv) with a pulse duration in the range of between, optionally, 1 s and 20 min, 2 s and 10 min, 3 s and 5 min, 5 s and 60 s, 10 s and 50 s, 20 s and 40 s, optionally about 30 s;
(v) for a total time period in the range of between, optionally, 5 seconds to 12 hours, 10 seconds to 6 hours, 30 seconds to 3 hours, 1 minute to 1.5 hours, 2 minutes to 1 hour, 3 minutes to 40 minutes, 4 minutes to 30 minutes, 5 minutes to 25 minutes, optionally about 15 minutes; and/or
(vi) at a temperature in the range of between, optionally, 1° C. and 42° C., 1° C. and 37° C., 1° C. and 35° C., 1° C. and 30° C., 1° and 25°, 1° C. and 20° C., 1° C. and 15° C., 1° C. and 10° C., 2° C. and 6° C., 3° C. and 5° C., and optionally at about 4° C.

8. The method of claim 1, wherein the electromagnetic stimulation in (a) is conducted by applying a continuous or pulsed, optionally pulsed, electromagnetic radiation:
(i) at a frequency in the range of between, optionally, 20 kHz and 500 kHz, 30 kHz and 450 kHz, 80 kHz and 400 kHz, 130 kHz and 350 kHz, 180 kHz and 300 kHz, 200 kHz and 280 kHz, 220 kHz and 260 kHz, optionally about 240 kHz;
(ii) with an energy output in the range of between, optionally, 54 joule per pulse and 96 joule per pulse, 55 joule per pulse and 90 joule per pulse, 55 joule per pulse and 80 joule per pulse, 55 joule per pulse and 70 joule per pulse, 55 joule per pulse and 65 joule per pulse, optionally about 60 joule per pulse;
(iii) with a pulse duration in the range of between 10 μs and 120 μs, 20 μs and 100 μs, 30 μs and 80 μs, 40 μs and 60 μs, optionally about 50 μs;
(iv) with a pulse repetition rate in the range of between, optionally, 0.1 Hz and 20 Hz, 0.5 Hz and 15 Hz, 0.6 Hz and 10 Hz, 0.8 Hz and 5 Hz, 1 Hz and 3 Hz, optionally about 2 Hz;
(v) for a total time period in the range of between, optionally, 5 seconds to 12 hours, 10 seconds to 6 hours, 30 seconds to 3 hours, 1 minute to 1.5 hours, 2 minutes to 1 hour, 3 minutes to 40 minutes, 4 minutes to 30 minutes, 5 minutes to 25 minutes, optionally about 15 minutes; and/or
(vi) at a temperature in the range of between, optionally, 1° C. and 42° C., 1° C. and 37° C., 1° C. and 35° C., 1° C. and 30° C., 1° and 25°, 1° C. and 20° C., 1° C. and 15° C., 1° C. and 10° C., 2° C. and 6° C., 3° C. and 5° C., and optionally at about 4° C.

9. The method of claim 1, wherein the non-mammalian cells, prior to (a), are or have been subjected to a fermentation.

10. A method of fermenting natural raw material comprising EVs, the method comprising:
(A) obtaining non-mammalian cells by the method of claim 1; and
(B) subjecting the cells to a fermentation;
wherein optionally the fermentation comprises:
(i) admixing the non-mammalian cells with:
an aqueous salt solution; and/or
at least one microorganism selected from the group consisting of microbiome-associated bacteria, yeasts and molds, or any combination thereof; and/or
a carbohydrate substrate selected from the group consisting of fructose, glucose, galactose, maltose and lactose, or any combination thereof;
and/or
(ii) incubating, under substantially anaerobic atmospheric conditions, for a sufficient amount of time to allow the fermentation to proceed;
(C) optionally, collecting the EVs obtained from (B).

11. The EVs obtainable or that have been obtained by the method of claim 1.

12. The EVs from ginger cells, optionally from ginger rhizome cells, wherein:
said EVs comprise one or more miRNAs corresponding to member(s) of each of the ginger miRNA families MIR319, MIR159, MIR396, MIR168, MIR156 and MIR164, and wherein the one or more miRNAs corresponding to each of the families MIR319 and MIR159 are comprised at a higher abundance as compared to the one or more miRNAs corresponding to each of the families MIR396, MIR168, MIR156 and MIR164; and/or
said EVs comprise at least one miRNA from each of the following groups (i) to (vi): (i) SEQ ID NOs: 1-13; (ii) SEQ ID NOs: 14-17; (iii) SEQ ID NOs: 18-24; (iv) SEQ ID NOs: 25-30; (v) SEQ ID NOs: 31-44; and (vi) SEQ ID NOs: 45-30, wherein the total amount of the miRNA(s) corresponding to each of the groups (ii) and (v) is higher relative to the total amount of the miRNA(s) corresponding to each of the groups (i), (ii), (iv), and (vi).

13. The use of the EVs of claim 11 in the manufacture of a beverage, a non-beverage food, a dietary supplement, a drug and/or a cosmetic or personal care product.

14. (canceled)

15. A method for the treatment of a pathological condition selected from the group consisting of a cancerous disease, a cardiovascular disease, a neurodegenerative disease, a metabolic disease, an inflammatory disease, an immune disease and an infectious disease.

16. The method of claim 15, wherein administration of the Evs
stimulating and/or strengthening the immune defense;
promoting hematopoiesis;
treating inflammation;
promoting health and/or longevity;
enhancing mitochondrial function, optionally for enhancing mitochondrial ATP synthesis;
anti-aging and/or skin regeneration;
reducing oxidative stress, optionally oxidative stress and inflammation in skin pathologies;
reducing oxidative-stress-mediated cell damage, optionally skin damage;
treating and/or preventing neuro-degeneration;
treating and/or preventing a metabolic disorder, optionally selected from diabetes, hypertension, obesity and arteriosclerosis;
treating and/or preventing an acute or chronic respiratory disease (e.g., a lung disorder), optionally selected from asthma and chronic obstructive pulmonary disease (COPD);
I treating and/or preventing an eye disorder, optionally selected from astigmatism, dry eyes, and conjunctivitis;
treating and/or preventing a skin disease, optionally selected from atopic eczema and open wounds;
treating and/or preventing an (auto-) immune disorder and/or an allergy;
treating and/or preventing a joint disease;
improving cell function and/or organ function;
promoting (gastro-) intestinal (GI) health and/or amelioration of (gastro-) intestinal (GI) illness;
treating and/or preventing neoplasm;
as an anti-microbial agent;
for promoting hair growth and/or preventing hair loss;
treating and/or preventing an erectile dysfunction and/or for increasing sexual potency;
enhancing physical or mental performance; and/or
a method of diagnosis in vivo and/or prognosis in vivo of a disease.


SEQUENCE LISTING (63 sequences)

SEQ ID NO: 1 - RNA, 21 nt, Zingiber officinale
  source 1..21: mol_type = other RNA; note = Conserved miRNA 'miR156' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
ctgacagaagagagtgagcac

SEQ ID NO: 2 - RNA, 19 nt, Zingiber officinale
  source 1..19: mol_type = other RNA; note = Conserved miRNA 'miR156_2' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
ttgacagaagatagagagc

SEQ ID NO: 3 - RNA, 21 nt, Zingiber officinale
  source 1..21: mol_type = other RNA; note = Conserved miRNA 'miR156a' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tgacagaagagagtgagcaca

SEQ ID NO: 4 - RNA, 20 nt, Zingiber officinale
  source 1..20: mol_type = other RNA; note = Conserved miRNA 'miR156a_2' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tgacagaagagagagagcac

SEQ ID NO: 5 - RNA, 20 nt, Zingiber officinale
  source 1..20: mol_type = other RNA; note = Conserved miRNA 'miR156a-5p' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tgacagaagagagtgagcac

SEQ ID NO: 6 - RNA, 21 nt, Zingiber officinale
  source 1..21: mol_type = other RNA; note = Conserved miRNA 'miR156c' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
ttgacagaagagagagagcac

SEQ ID NO: 7 - RNA, 22 nt, Zingiber officinale
  source 1..22: mol_type = other RNA; note = Conserved miRNA 'miR156c-3p_2' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
gctcacttctctctctgtcagc

SEQ ID NO: 8 - RNA, 22 nt, Zingiber officinale
  source 1..22: mol_type = other RNA; note = Conserved miRNA 'miR156d-3p_4' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
gctctctatgcttctgtcatca

SEQ ID NO: 9 - RNA, 22 nt, Zingiber officinale
  source 1..22: mol_type = other RNA; note = Conserved miRNA 'miR156e-3p' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
gctcactgctctctctgtcatc

SEQ ID NO: 10 - RNA, 20 nt, Zingiber officinale
  source 1..20: mol_type = other RNA; note = Conserved miRNA 'miR156f-5p' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
ctgacagaagagagtgagca

SEQ ID NO: 11 - RNA, 22 nt, Zingiber officinale
  source 1..22: mol_type = other RNA; note = Conserved miRNA 'miR156j_1' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
gttgacagaagagagtgagcac

SEQ ID NO: 12 - RNA, 20 nt, Zingiber officinale
  source 1..20: mol_type = other RNA; note = Conserved miRNA 'miR156k_1' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tgacagaagagagggagcac

SEQ ID NO: 13 - RNA, 21 nt, Zingiber officinale
  source 1..21: mol_type = other RNA; note = Conserved miRNA 'miR156k_2' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
ttgacagaagagagtgagcac

SEQ ID NO: 14 - RNA, 21 nt, Zingiber officinale
  source 1..21: mol_type = other RNA; note = Conserved miRNA 'miR159' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tttggactgaagggagctcta

SEQ ID NO: 15 - RNA, 21 nt, Zingiber officinale
  source 1..21: mol_type = other RNA; note = Conserved miRNA 'miR159a.1' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tttggattgaagggagctctg

SEQ ID NO: 16 - RNA, 21 nt, Zingiber officinale
  source 1..21: mol_type = other RNA; note = Conserved miRNA 'miR159a_1' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tttggattgaagggagctcta

SEQ ID NO: 17 - RNA, 21 nt, Zingiber officinale
  source 1..21: mol_type = other RNA; note = Conserved miRNA 'miR159b-5p' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
agctgccgactcattcattca

SEQ ID NO: 18 - RNA, 21 nt, Zingiber officinale
  source 1..21: mol_type = other RNA; note = Conserved miRNA 'miR164a_1' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
cacgtgctccccttctccaac

SEQ ID NO: 19 - RNA, 21 nt, Zingiber officinale
  source 1..21: mol_type = other RNA; note = Conserved miRNA 'miR164a_2' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tggagaagcagggcacttgct

SEQ ID NO: 20 - RNA, 21 nt, Zingiber officinale
  source 1..21: mol_type = other RNA; note = Conserved miRNA 'miR164a_4' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tggagaagcagggcacgtgca

SEQ ID NO: 21 - RNA, 20 nt, Zingiber officinale
  source 1..20: mol_type = other RNA; note = Conserved miRNA 'miR164b' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tggagaagcagggcacgtgc

SEQ ID NO: 22 - RNA, 21 nt, Zingiber officinale
  source 1..21: mol_type = other RNA; note = Conserved miRNA 'miR164c_1' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tggagaagcagggtacgtgca

SEQ ID NO: 23 - RNA, 21 nt, Zingiber officinale
  source 1..21: mol_type = other RNA; note = Conserved miRNA 'miR164c-5p' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tggagaagcagggcacgtgcg

SEQ ID NO: 24 - RNA, 22 nt, Zingiber officinale
  source 1..22: mol_type = other RNA; note = Conserved miRNA 'miR164e-5p' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tggagaagcagggcacgtgcaa

SEQ ID NO: 25 - RNA, 20 nt, Zingiber officinale
  source 1..20: mol_type = other RNA; note = Conserved miRNA 'miR168' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tcgcttggtgcaggtcggga

SEQ ID NO: 26 - RNA, 22 nt, Zingiber officinale
  source 1..22: mol_type = other RNA; note = Conserved miRNA 'miR168-3p' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tcccgccttgcatcaactgaat

SEQ ID NO: 27 - RNA, 22 nt, Zingiber officinale
  source 1..22: mol_type = other RNA; note = Conserved miRNA 'miR168-5p' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tcgcttggtgcaggtcgggaac

SEQ ID NO: 28 - RNA, 21 nt, Zingiber officinale
  source 1..21: mol_type = other RNA; note = Conserved miRNA 'miR168a-3p' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
cccgccttgcatcaactgaat

SEQ ID NO: 29 - RNA, 21 nt, Zingiber officinale
  source 1..21: mol_type = other RNA; note = Conserved miRNA 'miR168a-5p' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tcgcttggtgcaggtcgggaa

SEQ ID NO: 30 - RNA, 19 nt, Zingiber officinale
  source 1..19: mol_type = other RNA; note = Conserved miRNA 'miR168b_1' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tcgcttggtgcaggtcggg

SEQ ID NO: 31 - RNA, 19 nt, Zingiber officinale
  source 1..19: mol_type = other RNA; note = Conserved miRNA 'miR319_1' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
ttggactgaagggagctcc

SEQ ID NO: 32 - RNA, 20 nt, Zingiber officinale
  source 1..20: mol_type = other RNA; note = Conserved miRNA 'miR319a' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
cttggactgaagggagctcc

SEQ ID NO: 33 - RNA, 21 nt, Zingiber officinale
  source 1..21: mol_type = other RNA; note = Conserved miRNA 'miR319a_1' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
ttggactgaagggagctccct

SEQ ID NO: 34 - RNA, 20 nt, Zingiber officinale
  source 1..20: mol_type = other RNA; note = Conserved miRNA 'miR319a-3p' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
ttggactgaagggagctccc

SEQ ID NO: 35 - RNA, 21 nt, Zingiber officinale
  source 1..21: mol_type = other RNA; note = Conserved miRNA 'miR319a-5p_1' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
agagcttccttcagtccactc

SEQ ID NO: 36 - RNA, 21 nt, Zingiber officinale
  source 1..21: mol_type = other RNA; note = Conserved miRNA 'miR319b_1' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
cttggactgaagggagctcct

SEQ ID NO: 37 - RNA, 20 nt, Zingiber officinale
  source 1..20: mol_type = other RNA; note = Conserved miRNA 'miR319b_2' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
ttggactgaagggtgctccc

SEQ ID NO: 38 - RNA, 21 nt, Zingiber officinale
  source 1..21: mol_type = other RNA; note = Conserved miRNA 'miR319b-3p' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
ttggactgaagggtgctccct

SEQ ID NO: 39 - RNA, 21 nt, Zingiber officinale
  source 1..21: mol_type = other RNA; note = Conserved miRNA 'miR319c' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
ttggactgaagggagctcctt

SEQ ID NO: 40 - RNA, 20 nt, Zingiber officinale
  source 1..20: mol_type = other RNA; note = Conserved miRNA 'miR319c_1' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
ttggactgaagggagctcct

SEQ ID NO: 41 - RNA, 21 nt, Zingiber officinale
  source 1..21: mol_type = other RNA; note = Conserved miRNA 'miR319c_2' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
cttggactgaagggagctccc

SEQ ID NO: 42 - RNA, 21 nt, Zingiber officinale
  source 1..21: mol_type = other RNA; note = Conserved miRNA 'miR319e_1' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tttggactgaagggagctcct

SEQ ID NO: 43 - RNA, 22 nt, Zingiber officinale
  source 1..22: mol_type = other RNA; note = Conserved miRNA 'miR319g_1' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
ttggactgaagggagctccttc

SEQ ID NO: 44 - RNA, 21 nt, Zingiber officinale
  source 1..21: mol_type = other RNA; note = Conserved miRNA 'miR319q' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tggactgaagggagctccttc

SEQ ID NO: 45 - RNA, 21 nt, Zingiber officinale
  source 1..21: mol_type = other RNA; note = Conserved miRNA 'miR396a-3p_1' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
gttcaataaagctgtgggaag

SEQ ID NO: 46 - RNA, 20 nt, Zingiber officinale
  source 1..20: mol_type = other RNA; note = Conserved miRNA 'miR396a-3p_4' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
gttcaataaagctgtgggaa

SEQ ID NO: 47 - RNA, 20 nt, Zingiber officinale
  source 1..20: mol_type = other RNA; note = Conserved miRNA 'miR396a-3p_5' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
ttcaataaagctgtgggaag

SEQ ID NO: 48 - RNA, 21 nt, Zingiber officinale
  source 1..21: mol_type = other RNA; note = Conserved miRNA 'miR396a-5p' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
ttccacagctttcttgaactg

SEQ ID NO: 49 - RNA, 20 nt, Zingiber officinale
  source 1..20: mol_type = other RNA; note = Conserved miRNA 'miR396b' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
ttccacagctttcttgaact

SEQ ID NO: 50 - RNA, 21 nt, Zingiber officinale
  source 1..21: mol_type = other RNA; note = Conserved miRNA 'miR396b-3p_1' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
gttcaataaagctgtgggaaa

SEQ ID NO: 51 - RNA, 21 nt, Zingiber officinale
  source 1..21: mol_type = other RNA; note = Conserved miRNA 'miR396b-3p_3' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
gttcaagaaagcccatggaaa

SEQ ID NO: 52 - RNA, 21 nt, Zingiber officinale
  source 1..21: mol_type = other RNA; note = Conserved miRNA 'miR396b-5p' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
ttccacagctttcttgaactt

SEQ ID NO: 53 - RNA, 22 nt, Zingiber officinale
  source 1..22: mol_type = other RNA; note = Conserved miRNA 'miR396c_3' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
ttccacaggctttcttgaactg

SEQ ID NO: 54 - RNA, 22 nt, Zingiber officinale
  source 1..22: mol_type = other RNA; note = Conserved miRNA 'miR396d' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
ctccacaggctttcttgaactg

SEQ ID NO: 55 - RNA, 21 nt, Zingiber officinale
  source 1..21: mol_type = other RNA; note = Conserved miRNA 'miR396e' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
ttccacagctttcttgaactg

SEQ ID NO: 56 - RNA, 21 nt, Zingiber officinale
  source 1..21: mol_type = other RNA; note = Conserved miRNA 'miR396e-5p_1' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tccacaggctttcttgaactg

SEQ ID NO: 57 - RNA, 22 nt, Zingiber officinale
  source 1..22: mol_type = other RNA; note = Conserved miRNA 'miR396f-5p' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tctccacaggctttcttgaact

SEQ ID NO: 58 - RNA, 21 nt, Zingiber officinale
  source 1..21: mol_type = other RNA; note = Conserved miRNA 'miR396g-3p' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
gttcaagaaagctgtggaaga

SEQ ID NO: 59 - RNA, 21 nt, Zingiber officinale
  source 1..21: mol_type = other RNA; note = Conserved miRNA 'miR396g-5p_1' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tcccacagctttattgaactg

SEQ ID NO: 60 - RNA, 20 nt, Zingiber officinale
  source 1..20: mol_type = other RNA; note = Conserved miRNA 'miR396h' in ginger (see Supplementary Table S2 of Xing H et al. Genome-wide investigation of microRNAs and expression profiles during rhizome development in ginger (Zingiber officinale Roscoe). BMC Genomics. 2022; 23(1):49)
tccacagctttcttgaactg

SEQ ID NO: 61 - RNA, 22 nt, Homo sapiens
  source 1..22: mol_type = other RNA; note = human miRNA 'hsa-miR-1269a' retrieved from miRTarBase (see Huang HY et al., Nucleic Acids Res. 2022;50(D1):D222-D230)
ctggactgagccgtgctactgg

SEQ ID NO: 62 - RNA, 22 nt, Homo sapiens
  source 1..22: mol_type = other RNA; note = human miRNA 'hsa-miR-1269b' retrieved from miRTarBase (see Huang HY et al., Nucleic Acids Res. 2022;50(D1):D222-D230)
ctggactgagccatgctactgg

SEQ ID NO: 63 - skipped